(19) United States
(12) Patent Application Publication
(10) Pub. No.: US 2026/0265758 A1
(43) Pub. Date: Sep. 10, 2026

(54) METHODS OF TREATING PULMONARY FIBROSIS

(71) Applicant: RAGE BIOTECH PTY LTD, Melbourne, Victoria (AU)

(72) Inventors: Merlin Christopher Thomas, Melbourne (AU); Raelene Jane Pickering, Melbourne (AU)

(73) Assignee: RAGE BIOTECH PTY LTD, Melbourne, Victoria (AU)

(21) Appl. No.: 19/481,475

(22) PCT Filed: May 17, 2024
(86) PCT No.: PCT/AU2024/050497

§ 371 Date: Nov. 4, 2025

(30) Foreign Application Priority Data
May 19, 2023 (AU) 2023901 558
Oct. 5, 2023 (WO) PCT/AU2023/050960

(51) Int. Cl. C12N 15/113; A61K 9/00; A61K 31/712; A61K 31/7125; A61P 11/00

(52) U.S. Cl. CPC C12N 15/1138; A61K 9/007; A61K 31/712; A61K 31/7125; A61P 11/00; C12N 2310/11; C12N 2310/315; C12N 2310/321; C12N 2310/322; C12N 2310/3231; C12N 2310/3513; C12N 2310/3519; C12N 2320/33

(57) ABSTRACT

The present invention relates to compositions, methods and kits for the treatment or prevention of a condition of the airway or lung involving fibrosis. In particular, the compositions, methods and kits are particularly useful, but not limited to, the treatment or prevention of pulmonary fibrosis, such as idiopathic pulmonary fibrosis. In one aspect, the present invention provides method of treating or preventing a condition of the airway or lung involving fibrosis in a subject in need thereof, the method comprising administering to the subject an antisense oligonucleotide (AON) that promotes the production of endogenous soluble RAGE, thereby treating or preventing a condition of the airway or lung involving fibrosis in a subject.

### FIELD OF THE INVENTION

The present invention relates to compositions, methods and kits for the treatment or prevention of a condition of the airway or lung involving fibrosis. In particular, the compositions, methods and kits are particularly useful, but not limited to, the treatment or prevention of pulmonary fibrosis, such as idiopathic pulmonary fibrosis.

### RELATED APPLICATIONS

This application claims the benefit of priority from Australian provisional application no. 2023901558 filed 19 May 2023, and International Application no. PCT/AU2023/050960 filed 5 Oct. 2023, the entire disclosures of which are incorporated herein by reference.

### BACKGROUND OF THE INVENTION

Lung fibrosis, also referred to as pulmonary fibrosis, is a serious medical condition that involves scarring of the lung tissue. This condition occurs when the alveoli and interstitial tissue of the lungs become inflamed and develop scars on the tissue in an attempt to repair themselves. Pulmonary fibrosis involves gradual exchange of normal lung parenchyma with fibrotic tissue (fibrous scar). The replacement of normal lung with scar tissue causes irreversible decrease in oxygen diffusion capacity. Currently, there is no cure or means by which to reverse this scarring of the lung tissue.

Pulmonary fibrosis can be caused by many conditions which includes chronic inflammatory processes (sarcoidosis, Wegener's granulomatosis), infections, environmental agents (asbestos, silica, exposure to certain gases), exposure to ionizing radiation (such as radiation therapy to treat tumors of the chest), smoke or inhaled tobacco products, chronic conditions (lupus), and certain medications (e.g. amiodarone, bleomycin, pingyangmycin, busulfan, methotrexate, and nitrofurantoin).

In a condition known as hypersensitivity pneumonitis, fibrosis of the lung can develop following a heightened immune reaction to inhaled organic dusts or occupational chemicals. This condition most often results from inhaling dust contaminated with bacterial, fungal, or animal products.

In some subjects, chronic pulmonary inflammation and fibrosis develop without an identifiable cause. Most of these subjects have a condition called idiopathic pulmonary fibrosis (IPF). IPF is a chronic progressive pulmonary fibrosis of unknown etiology.

There is a need for new or improved methods for the treatment and/or prevention of pulmonary fibrosis.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect, the present invention provides a method of treating or preventing a condition of the airway or lung involving fibrosis in a subject in need thereof, the method comprising administering to the subject an antisense oligonucleotide (AON) that promotes the production of endogenous soluble RAGE and/or reduces the production of membrane bound RAGE (mRAGE), thereby treating or preventing a condition of the airway or lung involving fibrosis in a subject.

In another aspect, the invention also provides a method of alleviating or ameliorating a symptom of a condition of the airway or lung involving fibrosis in a subject in need thereof, the method comprising administering to the subject in need thereof an antisense oligonucleotide (AON) that promotes the production of endogenous soluble RAGE and/or reduces the production of membrane bound RAGE (mRAGE), thereby alleviating or ameliorating a symptom of a condition of the airway or lung involving fibrosis in the subject.

In another aspect, the invention also provides use of an antisense oligonucleotide (AON) that promotes the production of endogenous soluble RAGE and/or reduces the production of membrane bound RAGE (mRAGE) in the manufacture of a medicament for the treatment or prevention of a condition of the airway or lung involving fibrosis in a subject in need thereof.

In another aspect, the invention also provide use of an antisense oligonucleotide (AON) that promotes the production of endogenous soluble RAGE and/or reduces the production of membrane bound RAGE (mRAGE) in the manufacture of a medicament for alleviating or ameliorating a symptom of a condition of the airway or lung involving fibrosis in a subject in need thereof.

In another aspect, the invention also provide an antisense oligonucleotide (AON) that promotes the production of endogenous soluble RAGE and/or reduces the production of membrane bound RAGE (mRAGE) for use in a method of treating or preventing a condition of the airway or lung involving fibrosis in a subject in need thereof, the method comprising administering to the subject in need thereof an antisense oligonucleotide (AON), thereby treating or preventing a condition of the airway or lung involving fibrosis in a subject.

In another aspect, the invention also provide an antisense oligonucleotide (AON) that promotes the production of endogenous soluble RAGE and/or reduces the production of membrane bound RAGE (mRAGE) for use in a method of alleviating or ameliorating a symptom of a condition of the airway or lung involving fibrosis in a subject in need thereof, the method comprising administering to the subject in need thereof an antisense oligonucleotide (AON), thereby alleviating or ameliorating a symptom of a condition of the airway or lung involving fibrosis in the subject.

In another aspect, the present invention provides a method for the treatment or prevention of a condition of the airway or lung involving fibrosis in a subject comprising the steps of
identifying a subject having a condition of the airway or lung involving fibrosis; and
administering to the subject in need thereof an antisense oligonucleotide (AON) that promotes the production of endogenous soluble RAGE and/or reduces the production of membrane bound RAGE (mRAGE),
   thereby treating or preventing a condition of the airway or lung involving fibrosis in the subject.

In another aspect, the present invention provides an antisense oligonucleotide (AON) that promotes the production of endogenous soluble RAGE and/or reduces the production of membrane bound RAGE (mRAGE) for use in a method for the treatment or prevention of a condition of the airway or lung involving fibrosis in a subject in need thereof, the method comprising the steps of
identifying a subject having a condition of the airway or lung involving fibrosis; and
administering to the subject in need thereof the antisense oligonucleotide (AON),
   thereby treating or preventing a condition of the airway or lung involving fibrosis in the subject.

In any aspect, the AON that promotes the production of endogenous soluble RAGE and/or reduces the production of membrane bound RAGE (mRAGE) is administered directly to the airway and/or lungs.

For any aspect of the invention, the administration to the airway and the lungs is via any route that allows an antisense oligonucleotide (AON) that promotes the production of endogenous soluble RAGE and/or reduces the production of membrane bound RAGE (mRAGE) to contact the airway, lung or a part thereof. For example, an antisense oligonucleotide (AON) that promotes the production of endogenous soluble RAGE and/or reduces the production of membrane bound RAGE (mRAGE) may be administered via any route such that there is improvement in a symptom of the disease or condition to be treated or prevented. Preferably, the route of administration allows exposure to the respiratory tract or lung parenchyma such as alveolar tissue with respiratory bronchioles, alveolar ducts and terminal bronchioles. Preferably, an antisense oligonucleotide (AON) that promotes the production of endogenous soluble RAGE and/or reduces the production of membrane bound RAGE (mRAGE) is formulated for inhalation or intranasal administration.

In any aspect or embodiment, the antisense oligonucleotide (AON) that promotes the production of endogenous soluble RAGE and/or reduces the production of membrane bound RAGE (mRAGE) is administered via inhalation or intranasal administration.

In another aspect, the invention provides a kit or article of manufacture comprising any an antisense oligonucleotide (AON) that promotes the production of endogenous soluble RAGE and/or reduces the production of membrane bound RAGE (mRAGE), and/or a pharmaceutical composition comprising an antisense oligonucleotide (AON) that promotes the production of endogenous soluble RAGE and/or reduces the production of membrane bound RAGE (mRAGE).

In any aspect or embodiment of the invention, the condition of the airway or lung involving fibrosis is pulmonary fibrosis. In any embodiment, the pulmonary fibrosis is idiopathic pulmonary fibrosis, familial pulmonary fibrosis, pulmonary fibrosis caused by sarcoidosis, pulmonary fibrosis caused by silicosis, pulmonary fibrosis caused by asbestosis, pulmonary fibrosis caused by coal worker's pneumoconiosis, pulmonary fibrosis caused by carbon pneumoconiosis, pulmonary fibrosis caused by hypersensitivity pneumonitides, pulmonary fibrosis caused by inhalation of inorganic dust, pulmonary fibrosis caused by an infectious agent, pulmonary fibrosis caused by inhalation of noxious gases, aerosols, chemical dusts, fumes or vapors, drug-induced interstitial lung disease.

In any aspect, a method or use of the invention may promote the production of endogenous soluble RAGE and/or reduce the production of membrane bound RAGE (mRAGE) in a subject, particularly the respiratory tract of the subject. In any embodiment, a method of the invention may promote the production of endogenous soluble RAGE and/or reduce the production of membrane bound RAGE (mRAGE) in one or more tissues of the respiratory tract, for example one or more of the tissues of the respiratory tract described herein.

In any aspect, an AON may promote the production of endogenous soluble RAGE by promoting the inclusion of exon 9b and/or the exclusion (e.g. skipping) of exon 10. Therefore, in any aspect, the AON may promote splicing in the RAGE pre-mRNA resulting in the inclusion of exon 9b and/or skipping of exon 10. For example, the AON may result in an increase of the level of RAGE_v1, RAGE_v6, RAGE_v8, RAGE_v9 RAGE_v10, RAGE_v15, RAGE_v18, and RAGE_v19 mRNA, preferably an increase in the level of RAGE_v1, RAGE_v6, RAGE_v8, RAGE_v9, RAGE_v10, RAGE_v15, RAGE_v18, and RAGE_v19 mRNA in one or more tissues of the respiratory tract.

In any aspect, the AON is administered to the total respiratory tract, the upper respiratory tract or the lower respiratory tract.

As used herein, the upper respiratory tract may include any one or more of the following regions: the nose and nasal passages, paranasal sinuses, the pharynx, and the portion of the larynx above the vocal folds (cords). Typically, the lower respiratory tract includes any one or more of the following regions: the portion of the larynx below the vocal folds, trachea, bronchi and bronchioles. The lungs can be included in the lower respiratory tract and include the respiratory bronchioles, alveolar ducts, alveolar sacs, and alveoli.

In any aspect, the AON is administered as an aerosol, a dry powder or as nasal drops. In any embodiment, the AON may be administered using a nasal spray pump, intranasal installation, intratrachael instillation, metered dose inhaler (MDI), dry powder inhaler (DPI), nebuliser (jet, ultrasonic mesh or vibrating mesh) or soft mist inhaler (SMI).

In any aspect, the AON is an AON of 10 to 50 nucleotides comprising a targeting sequence complementary to a region near or within an intron of the RAGE pre-mRNA. Alternatively, the AON is 10 to 50 nucleotides comprising a targeting sequence complementary or adjacent to a splice site of the RAGE pre-mRNA.

Because factors such as RNA secondary structure, competition between AONs and SR proteins, heterogeneous nuclear ribonucleoproteins (hnRNPs), and/or other elements that make up the spliceosome can affect AONs' action, AONs directed at the crucial acceptor or donor splice sites will not always alter splicing. Consequently, in any aspect of the invention, the AON is 10 to 50 nucleotides comprising a targeting sequence complementary or adjacent to cis-acting RNA elements in the pre-mRNA of RAGE that act as enhancers or silencers, that, when bound by an elements of the splicosome (e.g. protein-splicing factors, uRNA, lncRNA) modulates the splicing of a nearby exon.

In any aspect, the AON is 10 to 50 nucleotides comprising a targeting sequence complementary to RAGE pre-mRNA which modulates secondary structure of said mRNA to influence splice site selection.

In any aspect, the AON is an isolated or purified AON for inducing exclusion (also known as skipping) of one or more exonic sequences in the RAGE gene transcript or part thereof.

In any aspect, the AON is an isolated or purified AON for inducing retention of intronic sequences in the RAGE gene transcript or part thereof.

In any aspect, the AON comprises at least one modified nucleotide. Typically, the AON is chemically-modified to prevent degradation of the pre-mRNA-AON complex, including but not limited to phosphorodiamidate morpholino oligomers (PMO), 2′ O-methyl phosphorothioate oligonucleotides (2OMe), and 2′-O-methoxyethyl phosphorothioate oligonucleotides (2′-MOE), locked nucleic acid (LNA) modified AONs, thermostable twisted intercalating nucleic acid (TINA) and peptide nucleic acids (PNAs).

In any aspect, the AON comprises at least one modified nucleotide selected from the group consisting of: phosphorodiamidate morpholino oligomers (PMO), 2′ O-methyl oligonucleotides (2OMe), 2′-O-methoxyethyl oligonucleotides (2′-MOE), phosphorothioate oligonucleotides, locked nucleic acid (LNA) modified AONs, thermostable twisted intercalating nucleic acid (TINA) and peptide nucleic acids (PNAs).

In any aspect, the AONs may be conjugated to moieties to increase their delivery, including but not limited to cell-penetrating peptides (CPPs), vivo-morpholinos (VMO) or peptide phosphorodiamidate morpholino oligomers (PPMO).

In any aspect, the antisense oligonucleotide comprises, consists essentially of or consists of a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or 100% complementary to a target region of exon 10 of RAGE pre-mRNA over the entirety of the antisense oligonucleotide. Preferably, the 5′-most nucleotide of the AON is nucleotide position 88 or 114 of exon 10 or is between nucleotide positions 88 to 114 of exon 10. Preferably, the 5′-most nucleotide of the target region is nucleotide position 88 or 114 of exon 10 or is between nucleotide positions 88 to 114 of exon 10.

In any embodiment, the 5′-most nucleotide of the target region is position 88 or 113, or between nucleotide positions 88 to 113 of exon 10 of RAGE pre-mRNA.

In any embodiment, the 5′-most nucleotide of the target region is position 90 or 113, or between nucleotide positions 90 to 113 of exon 10 of RAGE pre-mRNA.

In any embodiment, the 5′-most nucleotide of the target region is position 88 or 108, or between nucleotide positions 88 to 108 of exon 10 of RAGE pre-mRNA.

In any embodiment, the 5′-most nucleotide of the target region is position 90 or 108, or between nucleotide positions 90 to 108 of exon 10 of RAGE pre-mRNA.

In any embodiment, the 5′-most nucleotide of the target region is position 88 or 95, or between nucleotide positions 88 to 95 of exon 10 of RAGE pre-mRNA.

In any embodiment, the target region is from nucleotide position 88 to 137 of exon 10 of RAGE pre-mRNA.

In any embodiment, the target region is from nucleotide position 88 to 107 of exon 10 of RAGE pre-mRNA.

In any embodiment, the target region is from nucleotide position 90 to 102 of exon 10 of RAGE pre-mRNA.

In any embodiment, the target region is from nucleotide position 95 to 119 of exon 10 of RAGE pre-mRNA.

In any embodiment, the target region is between nucleotide positions 108 to 132 of exon 10 of RAGE pre-mRNA.

In any embodiment, the target region is between nucleotide positions 113 to 137 of exon 10 of RAGE pre-mRNA.

In any aspect, the AON may be 8 to 40 nucleotides in length, 15 to 25 nucleotides in length or 18 nucleotides in length.

In any embodiment, the AON is selected from the group comprising the sequences set forth in any of Tables 1a-1d. Preferably, the AON is selected from the list comprising: SEQ ID NO: 1-31 and 33, for example, the AON is SEQ ID NO: 11, 18, 19, or 20 or a nucleotide sequence at least 85%, 90% or 95% identical thereto.

In any embodiment, the AON may be selected to be an AON capable of binding to a selected target site, wherein the target site is a putative mRNA splicing site selected from a splice donor site, splice acceptor site, splice enhancer sequences splice silencer sequences or sites that modulate the secondary structure of pre-mRNA. The target site may also include some flanking intronic sequences when the donor or acceptor splice sites are targeted.

More specifically, the AON may be selected from the group comprising of any one or more of SEQ ID NOs: 1-31 and 33 and/or the sequences set forth in any of Tables 1a-1d, and combinations or cocktails thereof. More preferably, the AON is SEQ ID NO: 11, 18, 19, or 20. The combination of AONs is preferably a combination of SEQ ID NO: 11 and 10, or SEQ ID NO: 11 and 13. This includes sequences which can hybridise to such sequences under stringent hybridisation conditions, sequences complementary thereto, sequences containing modified bases, modified backbones, and functional truncations or extensions thereof which possess or modulate pre-mRNA processing activity in a RAGE gene transcript.

In certain embodiments, AONs may be 100% complementary to the target sequence, or may include mismatches, e.g., to accommodate variants, as long as a hetero-duplex formed between the oligonucleotide and target sequence is sufficiently stable to withstand the action of cellular nucleases and other modes of degradation which may occur in vivo. Hence, certain oligonucleotides may have about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence.

The methods and uses of the invention extends also to a combination of two or more AONs capable of binding to a selected target to modulate alternative splicing of the RAGE pre-mRNA, including a construct comprising two or more such AONs. The constructs may be used together for a combined AON-based therapy. The combination of AONs is preferably a combination of SEQ ID NO: 11 and 10, or SEQ ID NO: 11 and 13.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Segmental broncho-alveolar lavage fluid (BALF) analysis. Different parts of the mini-bronchoscope for intra-bronchial administration in mice. Visualization of trachea bifurcation (blue box) leading to right main bronchus that (green box) branches into diaphragmatic, cardiac and azygous bronchi (yellow box) or left main bronchus (red box), as acquired with the built-in camera of the bronchoscope. Vehicle/bleomycin and ASO 6713 (3 mg/kg or 10 mg/kg) were delivered specifically into the left main bronchus (red box), while right side was not treated.

FIG. 2. Flow cytometry analysis of CD45+ cells in segmental BALF obtained 7 or 14 days following treatment with bleomycin or vehicle. Flow cytometry of segmental BALF and analysis of hematopoietic (CD45+) total cell numbers obtained from the left lobe of the lungs of mice at day 7 (inflammatory phase) (A) and day 14 (fibrotic phase) (B) following treatment with bleomycin or vehicle for the following groups: vehicle (control group), Bleomycin alone (fibrosis group), Bleomycin+3 mg/kg ASO 6713 (low dose group) and Bleomycin+10 mg/kg ASO 6713 (high dose group). Data shows Mean±SD; Doses are denoted+0 (saline/vehicle), +3, 3 mg/kg intralobar ASO 6713, +10, 10 mg/kg intralobar ASO 6713.

FIG. 3. Flow cytometry analysis of interstitial macrophages, CD4+ T cells and CD8+ T cells in segmental BALF obtained 7 days following treatment with bleomycin or vehicle. Flow cytometry of segmental BALF and analysis of total cell number for interstitial (non-alveolar) macrophages (A), CD4+ T cells (B) and CD8+ T cells (C) obtained from the left lobe of the mice 7 days (during the inflammatory phase) following treatment with bleomycin or vehicle for the following groups: vehicle (control group), Bleomycin alone (fibrosis group), Bleomycin+3 mg/kg ASO 6713 (low dose group) and Bleomycin+10 mg/kg ASO 6713 (high dose group). Data shows Mean±SD; Doses are denoted +0 (saline/vehicle), +3, 3 mg/kg intralobar ASO 6713, +10, 10 mg/kg intralobar ASO 6713.

FIG. 4. Flow cytometry analysis of B cells, eosinophils and neutrophils in segmental BALF obtained 7 days following treatment with bleomycin or vehicle. Flow cytometry of segmental BALF and analysis of total cell number for B cells (A), eosinophils (B) and neutrophils (C) obtained from the left lobe of the mice 7 days (during the inflammatory phase) following treatment with bleomycin or vehicle for the following groups: vehicle (control group), Bleomycin alone (fibrosis group), Bleomycin+3 mg/kg ASO 6713 (low dose group) and Bleomycin+10 mg/kg ASO 6713 (high dose group). Data shows Mean±SD; Doses are denoted +0 (saline/vehicle), +3, 3 mg/kg intralobar ASO 6713, +10, 10 mg/kg intralobar ASO 6713.

FIG. 5. Flow cytometry analysis of activated macrophages, interstitial macrophages, dendritic cells in segmental BALF obtained 14 days following treatment with bleomycin or vehicle. Flow cytometry of segmental BALF and analysis of total cell number for activated macrophages (A), interstitial (non-alveolar) macrophages (B) and dendritic cells (C) obtained from the left lobe of the mice 14 days (during the fibrotic phase) following treatment with bleomycin or vehicle for the following groups: vehicle (control group), Bleomycin alone (fibrosis group), Bleomycin+3 mg/kg ASO 6713 (low dose group) and Bleomycin+10 mg/kg ASO 6713 (high dose group). Data shows Mean±SD; Doses are denoted +0 (saline/vehicle), +3, 3 mg/kg intralobar ASO 6713, +10, 10 mg/kg intralobar ASO 6713.

FIG. 6. Flow cytometry analysis of CD4+ T cells, CD8+ cells and B cells in segmental BALF obtained 14 days following treatment with bleomycin or vehicle. Flow cytometry of segmental BALF and analysis of total cell number for CD4+ T cells (A), CD8+ T cells (B) and B cells (C) obtained from the left lobe of the mice 14 days (during the fibrotic phase) following treatment with bleomycin or vehicle for the following groups: vehicle (control group), Bleomycin alone (fibrosis group), Bleomycin+3 mg/kg ASO 6713 (low dose group) and Bleomycin+10 mg/kg ASO 6713 (high dose group). Data shows Mean±SD; Doses are denoted +0 (saline/vehicle), +3, 3 mg/kg intralobar ASO 6713, +10, 10 mg/kg intralobar ASO 6713.

FIG. 7. IL6 and CCL2 Inflammatory cytokine expression in segmental BALF obtained 14 days following treatment with bleomycin or vehicle. Quantitative measurement of the inflammatory cytokines IL6 (A) and CCL2 (B) by ELISA in segmental BALF of the following groups: vehicle (control group), Bleomycin alone (fibrosis group), Bleomycin+3 mg/kg ASO 6713 (low dose group) and Bleomycin+10 mg/kg ASO 6713 (high dose group). Data shows Mean±SD; Doses are denoted +0 (saline/vehicle), +3, 3 mg/kg intralobar ASO 6713, +10, 10 mg/kg intralobar ASO 6713.

FIG. 8. Lymphocytic nodules in the lungs of mice at day 7 or day 14 following treatment with bleomycin or vehicle. Lymphocytic nodule counts (n/field) obtained from the left lobe of the lungs of mice at day 7 (inflammatory phase) (A) and day 14 (fibrotic phase) (B) following treatment with bleomycin or vehicle for the following groups: vehicle (control group), Bleomycin alone (fibrosis group), Bleomycin+3 mg/kg ASO 6713 (low dose group) and Bleomycin+10 mg/kg ASO 6713 (high dose group). Data shows Mean±SD; Doses are denoted +0 (saline/vehicle), +3, 3 mg/kg intralobar ASO 6713, +10, 10 mg/kg intralobar ASO 6713.

FIG. 9. The severity of lung injury in the lungs of mice 14 days following treatment with bleomycin or vehicle. Lung severity was quantified according to the mouse-adapted Ashcroft score (Ashcroft T et al., Journal of clinical pathology, 1988; 41(4):467-70) at day 14 (fibrotic phase) following treatment with bleomycin or vehicle for the following groups: vehicle (control group), Bleomycin alone (fibrosis group), Bleomycin+3 mg/kg ASO 6713 (low dose group) and Bleomycin+10 mg/kg ASO 6713 (high dose group). Data shows Mean±SD; Doses are denoted +0 (saline/vehicle), +3, 3 mg/kg intralobar ASO 6713, +10, 10 mg/kg intralobar ASO 6713.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

For purposes of interpreting this specification, terms used in the singular will also include the plural and vice versa.

The present invention is based on the surprising finding that administration of an antisense oligonucleotide (AON) that that promotes the production of endogenous soluble RAGE and/or reduces the production of membrane bound RAGE (mRAGE) can prevent or treat pulmonary fibrosis. In particular, administration of an AON that promotes the production of endogenous soluble RAGE to the respiratory tract in a preclinical model of pulmonary fibrosis was able to inhibit inflammation and fibrosis.

### Fibrosis

The phrase ‘a condition of the airway or lung involving fibrosis’ or ‘a condition of the airway or lung having a fibrotic component’ includes any disease or condition where there is the formation or development of excess fibrous connective tissue (fibrosis) in the airway or lung thereby resulting in the development of scarred (fibrotic) tissue. This includes pulmonary fibrosis, lung fibrosis or Idiopathic pulmonary fibrosis (IPF). More precisely, pulmonary fibrosis is a chronic disease that causes swelling and scarring of the alveoli and interstitial tissues of the lungs. The scar tissue replaces healthy tissue and causes inflammation. This damage to the lung tissue causes stiffness of the lungs which subsequently makes breathing more and more difficult.

The term “pulmonary fibrosis” or “lung fibrosis” means the formation or development of excess fibrous connective tissue (fibrosis) in the lung thereby resulting in the development of scarred (fibrotic) tissue. More precisely, pulmonary fibrosis is a chronic disease that causes swelling and scarring of the alveoli and interstitial tissues of the lungs. The scar tissue replaces healthy tissue and causes inflammation. This chronic inflammation is, in turn, the prelude to fibrosis. This damage to the lung tissue causes stiffness of the lungs which subsequently makes breathing more and more difficult.

Pulmonary fibrosis is a complicated illness that can arise from many different causes which include microscopic damage to the lungs induced by inhalation of small particles (asbestos, ground stone, metal dust, particles present in cigarette smoke, silica dust, etc). Alternatively, pulmonary fibrosis may arise as a secondary effect of other diseases (autoimmune disease, viral or bacterial infections, etc). Certain drugs such as cytotoxic agents (e.g. bleomycin, busulfan and methotrexate); antibiotics (e.g. nitrofurantoin, sulfasalazine); antiarrhythmics (e.g. amiodarone, tocainide); anti-inflammatory medications (e.g. gold, penicillamine); illicit drugs (e.g. crack, cocaine, heroin); also can cause pulmonary fibrosis. However, when pulmonary fibrosis appears without a known cause, it is termed as “idiopathic” or idiopathic pulmonary fibrosis (IPF).

Pulmonary fibrotic disorders are thought to begin with acute injury to the pulmonary parenchyma, leading to chronic interstitial inflammation, then to fibroblast activation and proliferation, and finally progressing to the common endpoint of pulmonary fibrosis and tissue destruction. Current research indicates that inflammation is less important in IPF, which appears to be primarily a disorder of fibroblast activation and proliferation in response to some as yet unknown trigger(s). Broadly, the manifestations of fibrotic lung disease can be grouped as follows: they may be chronic, insidious, and slowly progressive; they may be sub-acute, with a resolving, remitting, relapsing, or progressive course; and they may be acute, with a fulminant, progressive, remitting, or resolving course. Disorders with chronic, insidious, and slowly progressive courses are those that clinically resemble IPF and usually share a common pathology (i.e., UIP). Many of the connective-tissue diseases (e.g. rheumatoid arthritis; CREST syndrome (calcinosis cutis, Raynaud's syndrome, esophageal motility disorder, sclerodactyly, and telangiectasia); syndrome/progressive systemic scleroderma; systemic lupus erythematosus; mixed connective-tissue disease; pneumoconioses (e.g. asbestosis, silicosis); chronic hypersensitivity pneumonitis; and drug-related pulmonary fibrosis (e.g. due to bleomycin) generally fit into this category.

Development of clinically apparent lung diseases related to occupational exposures (e.g. pneumoconiosis) generally occurs many years after the exposure. Radiation fibrosis often develops months to years after radiation exposure. A lag time of months or years can occur between the use of pulmonary toxic medications and the development of fibrotic disease. The effect can be dose-dependent (e.g. bleomycin). Pulmonary manifestations of connective-tissue disease may develop in advance of, coincident with, or many years after the onset of articular disease. Pulmonary sarcoidosis, although sometimes acute or sub-acute in onset, in some cases may present insidiously over time. Sub-acute presentations with a variable course are typified by cryptogenic organizing pneumonia (COP). COP often develops weeks or months after the onset of a flulike illness. The course is variable and may either spontaneously remit or progress. The disorder is thought to be very responsive to steroid therapy, although it may recur when steroids are withdrawn or tapered. In some cases, COP may progress to end-stage fibrotic lung disease. Disorders with an acute onset are typified by acute interstitial pneumonitis (AlP), which is an idiopathic form of severe lung injury. The histopathology is that of adult respiratory distress syndrome with diffuse alveolar damage. Patients present either with no antecedent history of lung disease or as part of an accelerated phase of underlying interstitial disease. Most patients progress rapidly to respiratory failure. Some patients may improve with steroids or other immunosuppressive therapy.

‘Idiopathic pulmonary fibrosis (IPF)’ is a specific manifestation of idiopathic interstitial pneumonia (IIP), a type of interstitial lung disease. Interstitial lung disease, also known as diffuse parenchymal lung disease (DPLD), refers to a group of lung diseases affecting the interstitium. Microscopically, lung tissue from IPF patients shows a characteristic set of histological features known as usual interstitial pneumonia (UIP). UIP is therefore the pathologic presentation of IPF.

The existence of, improvement in, treatment of or prevention of a condition of the airway or lung involving fibrosis, particularly pulmonary fibrosis/lung fibrosis or Idiopathic pulmonary fibrosis may be by any clinically or biochemically relevant method of the subject or a biopsy therefrom. For example, a parameter measured may be the presence or degree of fibrosis, the content of collagen, fibronectin, or another extracellular matrix protein, the proliferation rate of the cells or any extracellular matrix components in the cells or transdifferentiation of the cells to myofibroblasts.

RAGE pre-mRNA Alternate Splicing

Alternative splicing is recognized as an important layer of post-transcriptional gene regulation for the Receptor for Advanced Glycation End-products (RAGE). Although most RAGE is expressed in its full length isoform, a number of different coding isoforms are generated through alternative splicing (also known as splicoforms), including splicoforms with N-terminal truncations, C-terminal truncations, and splicoforms retaining intronic sequences. These different splicoforms may act as possible regulators of the full-length RAGE receptor either by competitive ligand binding or by displacing the full-length protein from binding partners. Over twenty splicoforms have been identified in different tissues such as lung, liver, kidney, smooth muscle, endothelial cells and brain.

The different RAGE gene splice variants have been named RAGE, RAGE_v1 to RAGE_v19 according to the Human Gene Nomenclature Committee and are described in Hudson et al., (2008) The FASEB Journal, 22: 1572-1580, the contents of which are incorporated in its entirety.

As used herein, “endogenous soluble RAGE” refers to a polypeptide that lacks any signalling elements and/or the transmembrane domain of full-length RAGE. Endogenous soluble RAGE may be encoded, and subsequently translated, by an alternatively spliced RAGE pre-mRNA resulting in the inclusion of exon 9b and/or exclusion (eg skipping) of exon 10, resulting in a premature stop and the complete loss of the trans-membrane and cytoplasmic domains. For example, endogenous soluble RAGE may be encoded by one or more of RAGE_v1, RAGE_v6, RAGE_v8, RAGE_v9 RAGE_v10, RAGE_v15, RAGE_v18, and RAGE_v19 mRNA, preferably RAGE_v1. Therefore, it will be appreciated that any AON as described herein that promotes splicing in the RAGE pre-mRNA resulting in the inclusion of exon 9b and/or exclusion (eg skipping) of exon 10 may be used in the methods of the invention to promote the production of “endogenous soluble RAGE” in the respiratory tract and blood of a subject.

For example, a “endogenous soluble RAGE” as described herein may be endogenous secretory RAGE (esRAGE) which is encoded by RAGE_v1 mRNA. esRAGE constitutes ~5% of circulating RAGE in humans. The skipping of exon 10 in esRAGE-type splicing is ascribed to the limitation of intron length in higher eukaryotes. Roughly 45 nucleotides must separate the 5′ splice site and branch point, and the minimum distance between the branch point and 3′ splice site appears to be approximately 18 nucleotides, respectively. Therefore introns shorter than 70 nucleotides are extremely rare in mammals and cannot be spliced out efficiently. When the esRAGE 5′ splice site in intron 9 is selected, the distance between this site and the 3′ splice site that borders exon 10 is 46 nucleotides, which is considerably shorter than the lower limit of the intron length. Therefore, the use of the downstream, esRAGE 5′ splice site of intron 9 and the inclusion of exon 10 would be mutually exclusive. Among the known splice variants analyzed, all variants that used the downstream esRAGE 5′ splice site in intron 9 skipped exon 10; in contrast, all variants that used the upstream RAGE 5′ splice site in intron 9 included exon 10. Thus, the available evidence indicates that the selection of either one of the two alternative 5′ splice sites in intron 9 couples with inclusion or exclusion of exon 10. The means of regulation of this splicing or an external means to modulate has been previously unknown.

Membrane bound RAGE may be wildtype or full length RAGE, or a protein that has resulted from normal splicing. In other words, membrane bound RAGE contains a transmembrane domain and a short (42 amino acid) cytosolic tail. Further, the membrane bound RAGE may be encoded by a mature RNA, post-splicing, that contains does not include exon 9b and/or that does include exon 10.

### Soluble Splicoforms of Receptor for Advanced Glycation End-Products (RAGE)

Alternative splicing is recognized as an important layer of post-transcriptional gene regulation for the Receptor for Advanced Glycation End-products (RAGE). Although most RAGE is expressed in its full length isoform, a number of different coding isoforms are generated through alternative splicing (also known as splicoforms), including splicoforms with N-terminal truncations, C-terminal truncations, and splicoforms retaining intronic sequences. These different splicoforms may act as possible regulators of the full-length RAGE receptor either by competitive ligand binding or by displacing the full-length protein from binding partners. Over twenty splicoforms have been identified in different tissues such as lung, liver, kidney, smooth muscle, endothelial cells and brain.

The different RAGE gene splice variants have been named RAGE, RAGE_v1 to RAGE_v19 according to the Human Gene Nomenclature Committee and as described in Hudson et al., (2008) The FASEB Journal, 22: 1572-1580, the contents of which are incorporated in its entirety. For example, (run on) retention of intron 9 (exon 9b) results in a premature stop and the complete loss of the trans-membrane and cytoplasmic domains generating a C-terminus truncated splicoform. For example, RAGE_v1 is C-terminus truncated splicoform generated by skipping of exon 10 and retention of intron 9 (exon 9b). Alternatively, in another example, an AON as described herein may increase RAGE_v10 mRNA levels in a tissue or sample by promoting splicing in the RAGE pre-mRNA that results in the skipping of exon 10 and exon 11.

The C-terminus truncated splicoforms as described herein lack any signalling elements or the transmembrane domain and translate a c-terminus truncated RAGE, such as endogenous secretory RAGE (esRAGE), that is able to act as a decoy receptor, competing with full length RAGE for ligands or increasing ligand clearance. In particular, esRAGE constitutes ~5% of circulating RAGE in humans.

Higher circulating levels of esRAGE are associated with improved health outcomes and longevity while lower esRAGE is associated with many disease states including but not limited to atherosclerosis, diabetes, the metabolic syndrome, cardiovascular mortality, anaemia, autism and various tumorigenic states. Treatment of diabetic mice with recombinant esRAGE reduces atherosclerosis, vascular inflammation, renal and retinal damage.

Aberrant splicing of RAGE (and therefore dysfunctional RAGE signalling) has been reported in diabetes, some cancers and Alzheimer's disease.

The skipping of exon 10 in esRAGE-type splicing is ascribed to the limitation of intron length in higher eukaryotes. Roughly 45 nucleotides must separate the 5′ splice site and branch point, and the minimum distance between the branch point and 3′ splice site appears to be approximately 18 nucleotides, respectively. Therefore introns shorter than 70 nucleotides are extremely rare in mammals and cannot be spliced out efficiently. When the esRAGE 5′ splice site in intron 9 is selected, the distance between this site and the 3′ splice site that borders exon 10 is 46 nucleotides, which is considerably shorter than the lower limit of the intron length. Therefore, the use of the downstream, esRAGE 5′ splice site of intron 9 and the inclusion of exon 10 would be mutually exclusive. Among the known splice variants analyzed, all variants that used the downstream esRAGE 5′ splice site in intron 9 skipped exon 10; in contrast, all variants that used the upstream RAGE 5′ splice site in intron 9 included exon 10. Thus, the available evidence indicates that the selection of either one of the two alternative 5′ splice sites in intron 9 couples with inclusion or exclusion of exon 10. The means of regulation of this splicing or an external means to modulate has been previously unknown.

### Antisense Oligonucleotides (AONs)

Antisense oligonucleotide (AON) are short, synthetic, antisense, modified strands of DNA or RNA that can selectively hybridise to pre-RNA/mRNA through Watson-Crick base pairing and selectively modulate the function of the target RNA.

The terms “AON” and “ASO” are both abbreviations of the term “antisense oligonucleotide” and are used interchangeably herein.

Antisense oligonucleotide (AON) are short, synthetic, antisense, modified strands of DNA or RNA that can selectively hybridise to pre-RNA/mRNA through Watson-Crick base pairing and selectively modulate the function of the target RNA.

When AONs are used to modulate alternative splicing of mRNA, they are often referred to as splice-switching oligonucleotides (SSO). In the present invention, the terms AON and SSO may be used interchangeably. SSOs base-pair with a pre-mRNA and disrupt the normal splicing repertoire of the transcript by blocking the RNA-RNA base-pairing or protein-RNA binding interactions that occur between components of the splicing machinery and the pre-mRNA. SSOs can induce “skipping” of selected exons and/or retention of intronic sequences to modulate the product of translation. This can be achieved by targeting splice sites directly or by targeting cis-acting sequences involved in enhancing or silencing splicing by modulating binding of specific proteins or altering secondary structure of the pre mRNA.

Therapeutic SSOs may be used for the treatment of genetic disorders, to skip faulty or misaligned sections allowing for the generation of internally deleted, but now functional protein as a therapy.

The AONs for use in the methods of the present invention selectively manipulate the alternative splicing pattern of RAGE pre-mRNA, resulting in the generation of either natural RAGE mRNA splicoforms that are either non-functional or that act as a decoy receptor to antagonise ligand dependent activation and ligand-independent transactivation of full length RAGE. Preferably, the AONs also reduce the production of membrane bound RAGE.

Notably, there are no common RAGE polymorphisms at these splice sites. The RAGE sequence is highly conserved. Therefore, personalisation or individualised sequence modification is not required, unlike the management of genetic disorders with exon skipping technologies.

According to any aspect of the methods of the invention, the AONs as described herein are capable of binding to a selected target on a RAGE gene transcript to modulate pre-mRNA splicing in a RAGE gene transcript or part thereof. Broadly, the AON may be an isolated or purified AON. By “isolated” is meant material that is substantially or essentially free from components that normally accompany it in its native state. For example, an “isolated polynucleotide” or “isolated oligonucleotide,” as used herein, may refer to a polynucleotide that has been purified or removed from the sequences that flank it in a naturally-occurring state, e.g., a DNA fragment that is removed from the sequences that are adjacent to the fragment in the genome. The term “isolating” as it relates to cells, refers to the purification of cells (e.g., fibroblasts, lymphoblasts) from a source subject (e.g., a subject with a polynucleotide repeat disease). In the context of DNA, mRNA or protein, “isolating” refers to the recovery of the DNA, mRNA or protein from a source, e.g., cells.

According to any aspect of the methods of the invention, the AONs as described herein promote splicing in the RAGE pre-mRNA resulting in the inclusion of exon 9b and/or exclusion (eg skipping) of exon 10, resulting in a premature stop and the complete loss of the trans-membrane and cytoplasmic domains. Therefore, the AONs as described herein may increase the level of RAGE_v1, RAGE_v6, RAGE_v8, RAGE_v9. RAGE_v10, RAGE_v15. RAGE_v18, and RAGE_v19 mRNA, preferably an increase in the level of RAGE_v1, RAGE_v6, RAGE_v8, RAGE_v9, RAGE_v10, RAGE_v5, RAGE_v18, and RAGE_v19 mRNA in a tissue or sample. For example, an AON as described herein may increase RAGE_v1 mRNA levels in a tissue or sample by promoting splicing in the RAGE pre-mRNA that results in the skipping of exon 10 and retention of intron 9 (exon 9b). In another example, an AON as described herein may increase RAGE_v10 mRNA levels in a tissue or sample by promoting splicing in the RAGE pre-mRNA that results in the skipping of exon 10 and exon 11.

An AON can be said to be “directed to” or “targeted against” a target sequence with which it hybridizes. In certain embodiments, the target sequence includes a region including a 3′ or 5′ splice site of a pre-processed mRNA, a branch point, or other sequences involved in the regulation of splicing, including splice enhancers and splice silencers and sites determining the secondary structure of RNA that influence splicing. The target sequence may be within an exon or within an intron or spanning an intron/exon junction.

In any aspect, the antisense oligonucleotide comprises, consists essentially of or consists of a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or 100% complementary to a target region of exon 10 of RAGE pre-mRNA over the entirety of the antisense oligonucleotide. Preferably, the 5′-most nucleotide of the AON is nucleotide position 88 or 114 of exon 10 or is between nucleotide positions 88 to 114 of exon 10. Preferably, the 5′-most nucleotide of the target region is nucleotide position 88 or 114 of exon 10 or is between nucleotide positions 88 to 114 of exon 10. Typically, the nucleotide position may be identical or equal to SEQ ID NO: 32, where the first nucleotide is position 1.

| | |
|---|---|
| Human RAGE | GCTCTGTGGGAGGATCAGGGCTGGGAACTCTAGCCCTGGCCCTGGGGATCC |
| Exon 10 | TGGGAGGCCTGGGGACAGCCGCCCTGCTCATTGGGGTCATCTTGTGGCAAA |
| (SEQ ID NO: 32) | GGCGGCAACGCCGAGGAGAGGAGAG |

In certain embodiments, the AON has sufficient sequence complementarity to a target RNA (i.e., the RNA for which splice site selection is modulated) to block a region of a target RNA (e.g., pre-mRNA) in an effective manner. In exemplary embodiments, such blocking of RAGE pre-mRNA serves to modulate splicing, either by masking a binding site for a spliceosomal protein that would otherwise modulate splicing and/or by altering the structure of the targeted RNA. In some embodiments, the target RNA is target pre-mRNA (e.g., RAGE gene pre-mRNA).

An AON having a sufficient sequence complementarity to a target RNA sequence to modulate splicing of the target RNA means that the AON has a sequence sufficient to trigger the masking of a binding site for a native protein that would otherwise modulate splicing and/or alters the three-dimensional structure of the targeted RNA.

Selected AONs can be made shorter, e.g., about 12 bases, or longer, e.g., about 50 bases, and include a small number of mismatches, as long as the sequence is sufficiently complementary to effect splice modulation upon hybridization to the target sequence, and optionally forms with the RNA a heteroduplex having a Tm of 45° C. or greater.

Preferably, the AON is selected from the group comprising SEQ ID NOS: 1-31 and 33 and/or the sequences set forth in any of Tables 1a-1d. More preferably, the AON is SEQ ID NO: 11, 18, 19, or 20.

In certain embodiments, the degree of complementarity between the target sequence and AON is sufficient to form a stable duplex. The region of complementarity of the AONs with the target RNA sequence may be as short as 8-11 bases, but can be 12-15 bases or more, e.g., 10-50 bases, 10-40 bases, 12-30 bases, 12-25 bases, 15-25 bases, 12-20 bases, or 15-20 bases, including all integers in between these ranges. An AON of about 16-17 bases is generally long enough to have a unique complementary sequence. In certain embodiments, a minimum length of complementary bases may be required to achieve the requisite binding Tm, as discussed herein.

In certain embodiments, oligonucleotides as long as 50 bases may be suitable, where at least a minimum number of bases, e.g., 10-12 bases, are complementary to the target sequence. In general, however, facilitated or active uptake in cells is optimized at oligonucleotide lengths of less than about 30 bases. For phosphorodiamidate morpholino oligomer (PMO) AONs described further herein, an optimum balance of binding stability and uptake generally occurs at lengths of 18-25 bases. Included are AONs (e.g., PMOs, PMO-X, PNAs, LNAs, TINA, 2′-OMe) that consist of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 bases.

In certain embodiments, AONs may be 100% complementary to the target sequence, or may include mismatches, e.g., to accommodate variants, as long as a heteroduplex formed between the oligonucleotide and target sequence is sufficiently stable to withstand the action of cellular nucleases and other modes of degradation which may occur in vivo. Hence, certain oligonucleotides may have about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence.

Mismatches, if present, are typically less destabilizing toward the end regions of the hybrid duplex than in the middle. The number of mismatches allowed will depend on the length of the oligonucleotide, the percentage of G:C base pairs in the duplex, and the position of the mismatch(es) in the duplex, according to well understood principles of duplex stability. Although such an AON is not necessarily 100% complementary to the target sequence, it is effective to stably and specifically bind to the target sequence, such that splicing of the target pre-RNA is modulated.

The stability of the duplex formed between an AON and a target sequence is a function of the binding Tm and the susceptibility of the duplex to cellular enzymatic cleavage. The Tm of an oligonucleotide with respect to complementary-sequence RNA may be measured by conventional methods, such as those described by Hames et al., Nucleic Acid Hybridization, IRL Press, 1985, pp. 107-108 or as described in Miyada C. G. and Wallace R. B., 1987, Oligonucleotide Hybridization Techniques, Methods Enzymol. Vol. 154 pp. 94-107. In certain embodiments, AONs may have a binding Tm, with respect to a complementary-sequence RNA, of greater than body temperature and preferably greater than about 45° C. or 50° C. Tm's in the range 60-80° C. or greater are also included.

Additional examples of variants include AONs having about or at least about 70% sequence identity or homology, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity or homology, over the entire length of any of SEQ ID NOS: 1-31 and/or the sequences set forth in any of Tables 1a-1d. More preferably, the AON is SEQ ID NO: 11, 18, 19, or 20.

The modification of pre-mRNA splicing preferably induces “skipping”, or the removal of one or more exons or retention of introns of the mRNA. The resultant protein is preferably of a shorter length when compared to the parent full-length RAGE protein due to either internal truncation or premature termination. Preferably, the resultant protein has a C-terminal truncation. These truncated RAGE proteins may be termed splicoforms of the full length RAGE protein.

The remaining exons of the mRNA generated may be in-frame and produce a shorter protein with a sequence that is similar to that of the parent full length protein, except that it has an internal truncation in a region between the original 3′ and 5′ ends. In another possibility, the exon skipping may induce a frame shift that results in a protein wherein the first part of the protein is substantially identical to the parent full length protein, but wherein the second part of the protein has a different sequence (e.g. a nonsense sequence) due to a frame-shift. Alternatively, the exon skipping may induce the production of a prematurely terminated protein due to a disruption of the reading frame and presence of a premature termination of translation. The prematurely terminated protein may be the result of mRNA that is prematurely terminated (e.g. skipping of exons 10 and/or 11) or may be the result of a run on into an intron (e.g. RAGE 9b) or missense skip which provides an mRNA that contains the exon 10 and/or 11 mRNA, but which does not provide expression of the protein encoded by these exons.

Skipping individual exons of exons 1 to 9 will preferably disrupt the reading frame of the RAGE transcript. This will lead to increased degradation of RNA through nonsense mediated decay.

Skipping individual exons of exons 1 to 11 will preferably keep the reading frame intact. This will preferably lead to translation into an internally truncated protein. The truncated protein or RAGE mRNA splicoform may have a completely ablated function, may have a reduced function or act as a decoy receptor.

Preferably, these truncated, nonsense or prematurely terminated proteins are lacking one or more functional domains involved the induction of intracellular signalling pathways by RAGE ligands or non-ligand-dependent transactivation of RAGE by collocated GPCRs. For example, Exon 10 encodes a transmembrane domain and removing this exon may generate a soluble RAGE protein, which could potentially act as a soluble decoy or competitive antagonist of ligand induced signalling via RAGE. Truncated, nonsense or prematurely terminated proteins may further lack an attachment or binding site for other factors, removal of which may lead to a reduction in interaction of the RAGE protein with relevant signalling pathways.

Alternatively, the removal of one or more exons may lead to misfolding of the RAGE protein and a reduction in the ability of the protein to be successfully transported through the membrane.

The presence of internally truncated proteins (i.e. proteins lacking the amino acids encoded by one or more exons) is preferable. If the RAGE protein is inhibited, there may be problems with elevation of RAGE transcription as the body tries to compensate for the reduction in the total amount of RAGE protein. In contrast, the presence of an internally truncated protein (preferably lacking one or more of the features of the complete RAGE protein), should be sufficient to prevent elevated transcription, but still provide a therapeutic advantage due to a reduction in the total amount of functional RAGE protein.

The AON induced exon skipping as described herein need not completely or even substantially ablate the function of the RAGE protein. Preferably, the modulation of alternative splicing via the exon skipping process results in a reduced or compromised functionality of the RAGE protein.

The different isoforms of RAGE produced using different skipping strategies could result in proteins with ablated or reduced signalling activity that could preferably be used to treat or prevent different diseases associated with RAGE activity, such as neurodegenerative diseases, cancer, lung disorders, or inflammatory diseases. Alternative splicing strategies may form truncated proteins or proteins with reduced functions that can be preferably used as treatments for specific aspects, forms or progression of the diseases associated with RAGE expression and activity.

The skipping process using AONs may exclude (skip) an individual exon, or may result in skipping two or more exons at once.

The skipping process using AONs may include retention of intronic sequences with or without directly skipping one or more exons.

The AONs for use in the methods of the present invention may be a combination of two or more AONs capable of binding to a selected target to induce exon exclusion in a RAGE gene transcript. The combination may be a cocktail of two or more AONs and/or a construct comprising two or more or two or more AONs joined together.

TABLE 1a
Sequence of AONs for modulation of alternative splicing in human RAGE
Exon 9

| SEQ ID NO | AON# | Name | Sequence (5′ to 3′) |
|---|---|---|---|
| 1 | 3668 | H-AGERex9A(−10+15) | CCUCCUCGCCUGGUUCUGGAAGACA |
| 2 | 3669 | H-AGERex9A(+02+21) | UUGGCCCCUCCUCGCCUGGU |
| 3 | 3670 | H-AGERex9A(+08+27) | CUGCAGUUGGCCCCUCCUCG |
| 4 | 3671 | H-AGERex9D(+07−13) | UCAAACCCCUCACCUGCAGU |
| 5 | 4103 | H-AGERex9A(+3+27) | CCUGCAGUUGGCCCCUCCUCGCCUG |
| 6 | 4104 | H-AGERex9A(+10+27) | CCCCUCACCUGCAGUUGGCCCCUCC |
| 7 | 4105 | H-AGERex9A(+8+25) | GCAGUUGGCCCCUCCUC |
| 8 | 4106 | H-AGERex9D(+22−3) | CACCUGCAG UUG GCCCCUCCUCGCC |

TABLE 1b
Sequence of AONs for modulation of alternative splicing in human RAGE
Exon 10

| SEQ ID NO | AON# | Name | Sequence (5′ to 3′) |
|---|---|---|---|
| 9 | 3777 | AGER_H10A(−10+15) | AUCCUCCCACAGAGCCUGUACGGAG |
| 10 | 3778 | AGER_H10A(−5+20) | CCCUGAUCCUCCCACAGAGCCUGUA |
| 11 | 3779 | AGER_H10A(+90+114) | GGCGUUGCCGCCUUUGCCACAAGAU |
| 12 | 3780 | AGER_H10D(+20−5) | CUCACCUCUCCUCUCCUCGGCGUUG |
| 13 | 3781 | AGER_H10D(+15−10) | CUCCACUCACCUCUCCUCUCCUCGG |
| 14 | 82 | AGER_H10A (+60+79) | AGCAGGGCGGCUGUCCCCAG |
| 15 | 83 | AGER_H10A (+75+99) | GCCACAAGAUGACCCCAAUGAGCAG |
| 16 | 84 | AGER_A10 (+80+104) | CCUUUGCCACAAGAUGACCCCAAUG |
| 17 | 85 | AGER_A10 (+85+109) | UGCCGCCUUUGCCACAAGAUGACCC |
| 18 | 87 | AGER_H10(+95+119) | UCCUCGGCGUUGCCGCCUUUGCCAC |
| 19 | 90 | AGER_H10A(+90+102) | UUUGCCACAAGAU |
| 20 | 93 | AGER_H10A(+88+107) | CCGCCUUUGCCACAAGAUGA |

TABLE 1c
Sequence of AONs for modulation of alternative splicing in human RAGE
Intron 9

| SEQ ID NO | AON# | Name | Sequence (5′ to 3′) |
|---|---|---|---|
| 21 | 88 | AGER_H10A(−75−51) | UUUCUUGUUGACCAUCCCCCCAGUC |
| 22 | 89 | AGER_H10A(−86−62) | CCAUCCCCCCAGUCACAUGUGUUGG |

TABLE 1d
Sequence of AONs for modulation of alternative splicing in murine RAGE

| SEQ ID NO | AON# | Name | Sequence (5′ to 3′) |
|---|---|---|---|
| 23 | 3672 | M-AgerEx9A(−10+15) | CCUCCUCGCCUGGUUCUGGAAGACA |
| 24 | 3673 | M-AgerEx9A(+02+21) | CUGGCCCCUCAUCGCCGGUU |
| 25 | 3674 | M-AgerEx9A(+08+27) | CUUCAGCUGGCCCCUCAUCG |
| 26 | 3675 | M-AgerEx9D(+07−13) | UCCAGUCCCUCACCUUCAGC |
| 27 | m3779 | M-AgerEx10A(+90+114) | UGGGUUGUCGUUUUCGCCACAGGAU |
| | (m79) | | |
| 28 | m97 | M-AgerEx10A(+48,+70) | ACUACUCCCAGGCCUCCCAGGAU |
| 29 | m98 | M-AgerEx10A(+56+79) | GCAGGGCUACUACUCCCAGGCA |
| 30 | m101 | M-AgerEx10D(−15+10) | UCUCACUCACCUCUCCUCACGCCUG |
| 31 | m102 | M-AgerEx10A(−5+20) | CCCAGACUCACCCACAGAGCCUGUA |
| 33 | 6713 | M-AgerEx10A(+90+107) | TCGTTTTCGCCACAGGAT |

More specifically, the AON for use in the methods of the present invention may be selected from those set forth in any of Tables 1a-1d. The sequences are preferably selected from the group consisting of any one or more of any one or more of SEQ ID NOs: 1-31 and 33, and combinations or cocktails thereof. More preferably, the AON is SEQ ID NO: 11, 18, 19, or 20. The combination of AONs is preferably a combination of SEQ ID NO: 11 and 10, or SEQ ID NO: 11 and 13. This includes sequences which can hybridise to such sequences under stringent hybridisation conditions, sequences complementary thereto, sequences containing modified bases, modified backbones, and functional truncations or extensions thereof which possess or modulate pre-mRNA processing activity in a RAGE gene transcript.

In the context of the AONs for use in the methods of the present invention, those skilled in the art will recognise that both U and T residues are capable of binding A and are therefore interchangeable in any of the AON sequences disclosed herein. Accordingly, in any AON sequence disclosed herein, any T may be a U and/or any U may be a T. These U to T or T to U substitutions produce equally potent AONs for use in the methods of the present invention.

The oligomer and the DNA, cDNA or RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or pairing such that stable and specific binding occurs between the oligomer and the DNA, cDNA or RNA target. It is understood in the art that the sequence of an AON need not be 100% complementary to that of its target sequence to be specifically hybridisable. An AON is specifically hybridisable when binding of the compound to the target DNA or RNA molecule interferes with the normal function of the target DNA or RNA product, and there is a sufficient degree of complementarity to avoid non-specific binding of the AON to non-target sequences under conditions in which specific binding is desired, i.e., under physiological conditions in the case of in vivo assays or therapeutic treatment, and in the case of in vitro assays, under conditions in which the assays are performed.

Selective hybridisation may be under low, moderate or high stringency conditions, but is preferably under high stringency. Those skilled in the art will recognise that the stringency of hybridisation will be affected by such conditions as salt concentration, temperature, or organic solvents, in addition to the base composition, length of the complementary strands and the number of nucleotide base mismatches between the hybridising nucleic acids. Stringent temperature conditions will generally include temperatures in excess of 30° C., typically in excess of 37° C., and preferably in excess of 45° C., preferably at least 50° C., and typically 60° C.-80° C. or higher. Stringent salt conditions will ordinarily be less than 1000 mM, typically less than 500 mM, and preferably less than 200 mM. However, the combination of parameters is much more important than the measure of any single parameter. An example of stringent hybridisation conditions is 65° C. and 0.1×SSC (1×SSC=0.15 M NaCl, 0.015 M sodium citrate pH 7.0). Thus, the AONs for use in the methods of the present invention may include oligomers that selectively hybridise to the sequences provided in any of Tables 1a-1d, or SEQ ID NOs:1-31 and 33. More preferably, the AON is SEQ ID NO: 11, 18, 19, or 20.

It will be appreciated that the codon arrangements at the end of exons in structural proteins may not always break at the end of a codon, consequently there may be a need to delete more than one exon from the pre-mRNA to ensure in-frame reading of the mRNA. In such circumstances, a plurality of AONs may need to be selected by the method of the invention wherein each is directed to a different region responsible for inducing inclusion of the desired exon and/or intron. At a given ionic strength and pH, the Tm is the temperature at which 50% of a target sequence hybridizes to a complementary polynucleotide. Such hybridization may occur with “near” or “substantial” complementarity of the AON to the target sequence, as well as with exact complementarity.

Typically, selective hybridisation will occur when there is at least about 55% identity over a stretch of at least about 14 nucleotides, preferably at least about 65%, more preferably at least about 75% and most preferably at least about 90%, 95%, 98% or 99% identity with the nucleotides of the AON. The length of homology comparison, as described, may be over longer stretches and in certain embodiments will often be over a stretch of at least about nine nucleotides, usually at least about 12 nucleotides, more usually at least about 20, often at least about 21, 22, 23 or 24 nucleotides, at least about 25, 26, 27 or 28 nucleotides, at least about 29, 30, 31 or 32 nucleotides, at least about 36 or more nucleotides.

Thus, the AON for use in the methods of the present invention sequences preferably have at least 75%, more preferably at least 85%, more preferably at least 86, 87, 88, 89 or 90% homology to the sequences shown in the sequence listings herein. More preferably there is at least 91, 92, 93 94, or 95%, more preferably at least 96, 97, 98% or 99%, homology. Generally, the shorter the length of the AON, the greater the homology required to obtain selective hybridisation. Consequently, where an AON consists of less than about 30 nucleotides, it is preferred that the percentage identity is greater than 75%, preferably greater than 85, 86, 87, 88, 89, 90, 91, 92, 93, 94, 95%, 96, 97, 98% or 99% compared with the AONs set out in the sequence listings herein. Nucleotide homology comparisons may be conducted by sequence comparison programs such as the GCG Wisconsin Bestfit program or GAP (Deveraux et al., 1984, Nucleic Acids Research 12, 387-395). In this way sequences of a similar or substantially different length to those cited herein could be compared by insertion of gaps into the alignment, such gaps being determined, for example, by the comparison algorithm used by GAP.

The AONs for use in the methods of the present invention may have regions of reduced homology, and regions of exact homology with the target sequence. It is not necessary for an oligomer to have exact homology for its entire length. For example, the oligomer may have continuous stretches of at least 4 or 5 bases that are identical to the target sequence, preferably continuous stretches of at least 6 or 7 bases that are identical to the target sequence, more preferably continuous stretches of at least 8 or 9 bases that are identical to the target sequence. The oligomer may have stretches of at least 10, 11, 12, 13, 14, 15, 16, 17, 18, 19, 20, 21, 22, 23, 24, 25 or26 bases that are identical to the target sequence. The remaining stretches of oligomer sequence may be intermittently identical with the target sequence; for example, the remaining sequence may have an identical base, followed by a non-identical base, followed by an identical base. Alternatively (or as well) the oligomer sequence may have several stretches of identical sequence (for example 3, 4, 5 or 6 bases) interspersed with stretches of less than perfect homology. Such sequence mismatches will preferably have no or very little loss of splice switching activity.

The term “modulate” or “modulates” includes to “increase” or “decrease” one or more quantifiable parameters, optionally by a defined and/or statistically significant amount. The terms “increase” or “increasing,” “enhance” or “enhancing,” or “stimulate” or “stimulating” refer generally to the ability of one or AONs or compositions to produce or cause a greater physiological response (i.e., downstream effects) in a cell or a subject relative to the response caused by either no AON or a control compound. The terms “decreasing” or “decrease” refer generally to the ability of one or AONs or compositions to produce or cause a reduced physiological response (i.e., downstream effects) in a cell or a subject relative to the response caused by either no AON or a control compound.

Relevant physiological or cellular responses (in vivo or in vitro) will be apparent to persons skilled in the art, and may include increases in the exclusion of specific exons in a RAGE-coding pre-mRNA, decreases in the amount of RAGE-coding pre-mRNA or decreases in the expression of functional RAGE protein in a cell, tissue, or subject in need thereof. An “increased” or “enhanced” amount is typically a statistically significant amount, and may include an increase that is 1.1, 1.2, 2, 3, 4, 5, 6, 7, 8, 9, 10, 15, 20, 30, 40, 50 or more times (e.g., 500, 1000 times) (including all integers and decimal points in between and above 1, e.g., 1.5, 1.6, 1.7, 1.8) the amount produced by no AON (the absence of an agent) or a control compound. The term “reduce” or “inhibit” may relate generally to the ability of one or more AONs or compositions to “decrease” a relevant physiological or cellular response, such as a symptom of a disease or condition described herein, as measured according to routine techniques in the diagnostic art. Relevant physiological or cellular responses (in vivo or in vitro) will be apparent to persons skilled in the art, and may include reductions in the symptoms or pathology of a disease such as cancer, neurodegenerative diseases, lung disorders, and other inflammatory diseases. A “decrease” in a response may be statistically significant as compared to the response produced by no AON or a control composition, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers in between.

The length of an AON may vary, as long as it is capable of binding selectively to the intended location within the pre-mRNA molecule. The length of such sequences can be determined in accordance with selection procedures described herein. Generally, the AON will be from about 10 nucleotides in length, up to about 50 nucleotides in length. It will be appreciated, however, that any length of nucleotides within this range may be used in the method. Preferably, the length of the AON is between 10 and 40, 10 and 35, 15 to 30 nucleotides in length or 20 to 30 nucleotides in length, most preferably about 25 to 30 nucleotides in length. For example, the oligomer may be 20, 21, 22, 23, 24, 25, 26, 27, 28, 29 or 30 nucleotides in length.

As used herein, an “AON” refers to a linear sequence of nucleotides, or nucleotide analogs, that allows the nucleobase to hybridize to a target sequence in an RNA by Watson-Crick base pairing, to form an oligonucleotide:RNA heteroduplex within the target sequence. The terms “AON”, “AON”, “oligomer” and “antisense compound” may be used interchangeably to refer to an oligonucleotide. The cyclic subunits may be based on ribose or another pentose sugar or, in certain embodiments, a morpholino group (see description of morpholino oligonucleotides below). Also contemplated are peptide nucleic acids (PNAs), locked nucleic acids (LNAs), and 2′-O-Methyl oligonucleotides, 2′-O-Methoxyethyl oligonucleotides, among other antisense agents known in the art.

Included are non-naturally-occurring AONs, or “oligonucleotide analogs”, including AONs or oligonucleotides having (i) a modified backbone structure, e.g., a backbone other than the standard phosphodiester linkage found in naturally-occurring oligo- and polynucleotides, and/or (ii) modified sugar moieties, e.g., morpholino moieties rather than ribose or deoxyribose moieties. Oligonucleotide analogs support bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide (e.g., single-stranded RNA or single-stranded DNA). Preferred analogs are those having a substantially uncharged, phosphorus containing backbone.

One method for producing AONs is the methylation of the 2′ hydroxyribose position and the incorporation of a phosphorothioate backbone produces molecules that superficially resemble RNA but that are much more resistant to nuclease degradation, although persons skilled in the art of the methods of the invention will be aware of other forms of suitable backbones that may be useable in the objectives of the methods of the invention.

Increased splice-switching may also be achieved with alternative oligonucleotide chemistry. For example, the AON may be chosen from the list comprising: phosphoramidate or phosphorodiamidate morpholino oligomer (PMO); PMO-X; PPMO; peptide nucleic acid (PNA); a locked nucleic acid (LNA) and derivatives including alpha-L-LNA, 2′-amino LNA, 4′-methyl LNA and 4′-O-methyl LNA; ethylene bridged nucleic acids (ENA) and their derivatives; phosphorothioate oligomer; tricyclo-DNA oligomer (tcDNA); tricyclophosphorothioate oligomer; 2′O-Methyl-modified oligomer (2′-OMe); 2′-O-methoxy ethyl (2′-MOE); 2′-fluoro, 2′-fluroarabino (FANA); unlocked nucleic acid (UNA); thermostable twisted intercalating nucleic acid (TINA), hexitol nucleic acid (HNA); cyclohexenyl nucleic acid (CeNA); 2′-amino (2′-NH2); 2′-O-ethyleneamine or any combination of the foregoing as mixmers or as gapmers. To further improve the delivery efficacy, the above mentioned modified nucleotides are often conjugated with fatty acids/lipid/cholesterol/amino acids/carbohydrates/polysaccharides/nanoparticles etc. to the sugar or nucleobase moieties. These conjugated nucleotide derivatives can also be used to construct exon skipping AONs. Antisense oligonucleotide-induced splice modification of the human RAGE gene transcripts have generally used either oligoribonucleotides, PNAs, 2OMe or MOE modified bases on a phosphorothioate backbone. When alternative chemistries are used to generate the AONs for use in the methods of the invention, the uracil (U) of the sequences provided herein may be replaced by a thymine (T).

Included within the AONs for use in the methods of the present invention are non-naturally-occurring oligomers, or “oligonucleotide analogues,” including oligomers having (i) a modified backbone structure, e.g., a backbone other than the standard phosphodiester linkage found in naturally-occurring oligo- and polynucleotides, and/or (ii) modified sugar moieties, e.g., morpholino moieties rather than ribose or deoxyribose moieties. Oligomer analogues support bases capable of hydrogen bonding by Watson-Crick base pairing to standard polynucleotide bases, where the analogue backbone presents the bases in a manner to permit such hydrogen bonding in a sequence-specific fashion between the oligomer analogue molecule and bases in a standard polynucleotide (e.g., single-stranded RNA or single-stranded DNA). Preferred analogues are those having a substantially uncharged, phosphorus containing backbone.

Antisense oligonucleotides that do not activate RNase H can be made in accordance with known techniques (see, e.g., U.S. Pat. No. 5,149,797). Such AONs, which may be deoxyribonucleotide or ribonucleotide sequences, simply contain any structural modification which sterically hinders or prevents binding of RNase H to a duplex molecule containing the oligomer as one member thereof, which structural modification does not substantially hinder or disrupt duplex formation. Because the portions of the oligomer involved in duplex formation are substantially different from those portions involved in RNase H binding thereto, numerous AONs that do not activate RNase H are available. For example, such AONs may be oligomers wherein at least one, or all, of the inter-nucleotide bridging phosphate residues are modified phosphates, such as methyl phosphonates, methyl phosphorothioates, phosphoromorpholidates, phosphoropiperazidates boranophosphates, amide linkages and phosphoramidates. For example, every other one of the internucleotide bridging phosphate residues may be modified as described. In another non-limiting example, such AONs are molecules wherein at least one, or all, of the nucleotides contain a 2′ lower alkyl moiety (such as, for example, C₁-C₄, linear or branched, saturated or unsaturated alkyl, such as methyl, ethyl, ethenyl, propyl, 1-propenyl, 2-propenyl, and isopropyl). For example, every other one of the nucleotides may be modified as described.

Specific examples of preferred AONs for use in the methods of the present invention include oligomers containing modified backbones or non-natural inter-nucleoside linkages. As defined in this specification, oligomers having modified backbones include those that retain a phosphorus atom in the backbone and those that do not have a phosphorus atom in the backbone. For the purposes of this specification, and as sometimes referenced in the art, modified oligomers that do not have a phosphorus atom in their inter-nucleoside backbone can also be considered to be AONs.

In other preferred oligomer mimetics, both the sugar and the inter-nucleoside linkage, i.e., the backbone, of the nucleotide units are replaced with novel groups. The base units are maintained for hybridization with an appropriate nucleic acid target compound. One such oligomeric compound, an oligomer mimetic that has been shown to have excellent hybridization properties, is referred to as a peptide nucleic acid (PNA). In PNA compounds, the sugar-backbone of an oligomer is replaced with an amide containing backbone, in particular an aminoethylglycine backbone. The nucleo-bases are retained and are bound directly or indirectly to aza nitrogen atoms of the amide portion of the backbone.

Another preferred chemistry is the phosphorodiamidate morpholino oligomer (PMO) oligomeric compounds, which are not degraded by any known nuclease or protease. These compounds are uncharged, do not activate RNase H activity when bound to a RNA strand and have been shown to exert sustained splice modulation after in vivo administration (Summerton and Weller, Antisense Nucleic Acid Drug Development, 7, 187-197).

Modified oligomers may also contain one or more substituted sugar moieties. Oligomers may also include nucleobase (often referred to in the art simply as “base”) modifications or substitutions. Certain nucleobases are particularly useful for increasing the binding affinity of the oligomeric compounds as described herein. These include 5-substituted pyrimidines, 6-azapyrimidines, and N-2, N-6 and O-6 substituted purines, including 2-aminopropyladenine, 5-propynyluracil, 5-propynylcytosine and 5-methylcytosine substitutions have been shown to increase nucleic acid duplex stability by 0.6-1.2° C., even more particularly when combined with 2′-O-methoxyethyl sugar modifications.

The activity of AONs and variants thereof can be assayed according to routine techniques in the art. For example, splice forms and expression levels of surveyed RNAs and proteins may be assessed by any of a wide variety of well-known methods for detecting splice forms and/or expression of a transcribed nucleic acid or protein. Non-limiting examples of such methods include RT-PCR of spliced forms of RNA followed by size separation of PCR products, nucleic acid hybridization methods e.g., Northern blots and/or use of nucleic acid arrays; nucleic acid amplification methods; immunological methods for detection of proteins; protein purification methods; and protein function or activity assays.

RNA expression levels can be assessed by preparing mRNA/cDNA (i.e., a transcribed polynucleotide) from a cell, tissue or organism, and by hybridizing the mRNA/cDNA with a reference polynucleotide, which is a complement of the assayed nucleic acid, or a fragment thereof. cDNA can, optionally, be amplified using any of a variety of polymerase chain reaction or in vitro transcription methods prior to hybridization with the complementary polynucleotide; preferably, it is not amplified. Expression of one or more transcripts can also be detected using quantitative PCR to assess the level of expression of the transcript(s).

The AONs for use in the methods of the present invention may provide induced splice-switching of the RAGE gene transcript, clinically relevant oligomer chemistries and delivery systems to direct RAGE splice manipulation to therapeutic levels, promotion of the non-signalling decoy-receptor RAGE mRNA splicoform (e.g. RAGE_v1, RAGE_v6, RAGE_v8, RAGE_v9, RAGE_v0, RAGE_v5, RAGE_v18, and RAGE_v19 mRNA) can be achieved, and therefore may be used to treat a RAGE-related respiratory (pulmonary) disease or disorder.

The AONs for use in the methods of the present invention may be conveniently made through the well-known technique of solid phase synthesis. Equipment for such synthesis is sold by several vendors including, for example, Applied Biosystems (Foster City, Calif.). One method for synthesising oligomers on a modified solid support is described in U.S. Pat. No. 4,458,066.

Any other means for such synthesis known in the art may additionally or alternatively be employed. It is well known to use similar techniques to prepare oligomers such as the phosphorothioates and alkylated derivatives. In one such automated embodiment, diethyl-phosphoramidites are used as starting materials and may be synthesized as described by Beaucage, et al., (1981) Tetrahedron Letters, 22:1859-1862.

The AONs for use in the methods of the present invention are synthesised in vitro and do not include antisense compositions of biological origin, or genetic vector constructs designed to direct the in vivo synthesis of AONs.

### Administration, Dosage and Formulation

In any aspect, the methods of the present invention may be used to prevent or treat a condition of the airway or lung involving fibrosis in a subject in need thereof. Preferably, the condition of the airway or lung involving fibrosis is pulmonary fibrosis. Even more preferably, the condition of the airway or lung involving fibrosis is idiopathic pulmonary fibrosis, familial pulmonary fibrosis, pulmonary fibrosis caused by sarcoidosis, pulmonary fibrosis caused by silicosis, pulmonary fibrosis caused by asbestosis, pulmonary fibrosis caused by coal worker's pneumoconiosis, pulmonary fibrosis caused by carbon pneumoconiosis, pulmonary fibrosis caused by hypersensitivity pneumonitides, pulmonary fibrosis caused by inhalation of inorganic dust, pulmonary fibrosis caused by an infectious agent, pulmonary fibrosis caused by inhalation of noxious gases, aerosols, chemical dusts, fumes or vapors, drug-induced interstitial lung disease.

The term ‘respiratory’ refers to the process by which oxygen is taken into the body and carbon dioxide is discharged, through the bodily system including the nose, throat, larynx, trachea, bronchi and lungs.

As used herein, the upper respiratory tract may include the following regions: nose and nasal passages, paranasal sinuses, the pharynx, and the portion of the larynx above the vocal folds (cords). Typically, the lower respiratory tract includes any one of more of the following regions: portion of the larynx below the vocal folds, trachea, bronchi and bronchioles. The lungs can be included in the lower respiratory tract and include the respiratory bronchioles, alveolar ducts, alveolar sacs, and alveoli.

The term ‘respiratory disease’ or ‘respiratory condition’ refers to any one of several ailments that involve inflammation and affect a component of the respiratory system including the upper (including the nasal cavity, pharynx and larynx) and lower respiratory tract (including trachea, bronchi and lungs).

A symptom of respiratory disease may include cough, excess sputum production, a sense of breathlessness or chest tightness with audible wheeze. Exercise capacity may be quite limited. The impact of each of these conditions may also be measured by days of lost work/school, disturbed sleep, requirement for bronchodilator drugs, requirement for glucocorticoids including oral glucocorticoids.

The existence of, improvement in, treatment of or prevention of a respiratory disease may be determined by any clinically or biochemically relevant method of the subject or a biopsy therefrom. For example, a parameter measured may be the presence or degree of lung function, signs and symptoms of obstruction; exercise tolerance; night time awakenings; days lost to school or work; bronchodilator usage; inhaled corticosteroid (ICS) dose; oral (glucocorticoid) GC usage; need for other medications; need for medical treatment; hospital admission.

As used herein, ‘preventing’ or ‘prevention’ is intended to refer to at least the reduction of likelihood of the risk of (or susceptibility to) acquiring a disease or disorder (i.e., causing at least one of the clinical symptoms of the disease not to develop in a patient that may be exposed to or predisposed to the disease but does not yet experience or display symptoms of the disease). Biological and physiological parameters for identifying such patients are provided herein and are also well known by physicians.

The terms “treatment” or “treating” of a subject includes the application or administration of an AON as described herein with the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being.

The AONs as described herein may be are adapted to aid in the prophylactic or therapeutic treatment, prevention or amelioration of symptoms of a disease such as a RAGE expression related disease or pathology in a form suitable for delivery to a patient.

A positive response to therapy may also be prevention or attenuation of worsening of respiratory symptoms, described herein. This could be assessed by comparison of the mean change in disease score from baseline to end of study period based on lower respiratory symptom score (LRSS—symptoms of chest tightness, wheeze, shortness of breath and cough). Change from baseline lung function (peak expiratory flow PEF) could also be assessed and a positive response to therapy could be a significant attenuation in reduced PEF. For example, a placebo treated group would show a significant reduction in morning PEF of 15% at the peak of exacerbation whilst the treatment group would show a non-significant reduction in PEF less than 15% change from baseline.

Although the methods of the present invention finds application in humans, the methods of the invention is also useful for therapeutic veterinary purposes. The invention is useful for domestic or farm animals such as cattle, sheep, horses and poultry; for companion animals such as cats and dogs; and for zoo animals.

In certain embodiments, the AONs as described herein can be delivered by pulmonary or nasal routes (e.g., via nebulised saline incorporating the AONs). The highest endogenous expression of RAGE mRNA in the healthy human tissues is found in the lung and is accessible via the airways. Inhaled oligonucleotides are an emerging therapeutic modality for respiratory diseases. The airways are uniquely lined with pulmonary surfactants, which are primarily composed of zwitterionic lipids. These surfactant lipids possess cationic properties at the pH of the respiratory tract. When anionic oligonucleotides are inhaled, they tend to be adsorbed by the surfactants, resulting in reformulated particles that have been hypothesised to be efficiently taken up by bronchial and alveolar epithelial cells into the pulmonary cells. Of note, AONs have been shown to be able to withstand the nebulization process.

The AONs as described herein may be in compositions formulated for administration to the lower respiratory tract only. Limitation to the lower respiratory tract may be achieved by an amount, particularly volume and composition of form ie. particle size, physical form whether dry powder or solution droplet, of composition that would otherwise be administered to the upper respiratory tract. Alternatively, the AONs as described herein may be administered via a device that ensures retention in the lower respiratory tract only.

The AONs as described herein may be formulated for intranasal administration, including dry powder, sprays, mists, or aerosols. This may be particularly preferred for treatment of a respiratory infection.

Suitable formulations, wherein the carrier is a liquid, for administration, as for example, a nasal spray or as nasal drops, include aqueous or oily solutions of the active ingredient. Alternatively, the AONs as described herein may be provided as a dry powder and administered to the upper respiratory tract only as defined herein.

The selection of appropriate carriers depends upon the particular type of administration that is contemplated. For administration via the upper respiratory tract, e.g., the nasal mucosal surfaces, the compound can be formulated into a solution, e.g., water or isotonic saline, buffered or unbuffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2 (Remington's, Id. at page 1445). Of course, the ordinary artisan can readily determine a suitable saline content and pH for an innocuous aqueous carrier for nasal and/or upper respiratory administration.

Other ingredients, such as art known preservatives, colorants, lubricating or viscous mineral or vegetable oils, perfumes, natural or synthetic plant extracts such as aromatic oils, and humectants and viscosity enhancers such as, e.g., glycerol, can also be included to provide additional viscosity, moisture retention and a pleasant texture and odour for the formulation. For nasal administration of solutions or suspensions according to the methods of the invention, various devices are available in the art for the generation of drops, droplets and sprays. For example, an AON as described herein can be administered into the nasal passages by means of a simple dropper (or pipet) that includes a glass, plastic or metal dispensing tube from which the contents are expelled drop by drop by means of air pressure provided by a manually powered pump, e.g., a flexible rubber bulb, attached to one end.

A pharmaceutical composition may be formulated as inhaled formulations, including sprays, mists, or aerosols. This may be particularly preferred for treatment of a condition of the airway or lung involving fibrosis as described herein. The inhaled formulation may be for application to the upper (including the nasal cavity, pharynx and larynx) and lower respiratory tract (including trachea, bronchi and lungs). For inhalation formulations, the composition or combination provided herein may be delivered via any inhalation methods known to a person skilled in the art. Such inhalation methods and devices include, but are not limited to, metered dose inhalers with propellants such as HFA or propellants that are physiologically and environmentally acceptable. Other suitable devices are breath operated inhalers, multidose dry powder inhalers and aerosol nebulizers. Aerosol formulations for use in the subject method typically include propellants, surfactants and co-solvents and may be filled into conventional aerosol containers that are closed by a suitable metering valve. Different devices and excipients can be used depending on whether the application is to the upper (including the nasal cavity, pharynx and larynx) or lower respiratory tract (including trachea, bronchi and lungs) and can be determined by those skilled in the art. Further, processes for micronisation and nanoparticle formation for the preparation of AONs described herein for use in an inhaler, such as a dry powder inhaler, are also known by those skilled in the art.

Inhalant compositions may comprise liquid or powdered compositions containing the active ingredient that are suitable for nebulization and intrabronchial use, or aerosol compositions administered via an aerosol unit dispensing metered doses. Suitable liquid compositions comprise the active ingredient in an aqueous, pharmaceutically acceptable inhalant solvent such as isotonic saline or bacteriostatic water. The solutions are administered by means of a pump or squeeze-actuated nebulized spray dispenser, or by any other conventional means for causing or enabling the requisite dosage amount of the liquid composition to be inhaled into the patient's lungs. Suitable formulations, wherein the carrier is a liquid, for administration, as for example, a nasal spray or as nasal drops, include aqueous or oily solutions of the active ingredient. Examples of inhalation drug delivery devices are described in Ibrahim et al. Medical Devices: Evidence and Research 2015:8 131-139, are contemplated for use in the present invention.

The delivery of a therapeutically useful amount of AONs may be achieved by methods previously published. For example, intracellular delivery of the AON may be via a composition comprising an admixture of the AON and an effective amount of a block copolymer. An example of this method is described in US patent application US20040248833. Other methods of delivery of AONs to the nucleus are described in Mann C J et al. (2001) Proc, Natl. Acad. Science, 98(1) 42-47, and in Gebski et al. (2003) Human Molecular Genetics, 12(15): 1801-1811. A method for introducing a nucleic acid molecule into a cell by way of an expression vector either as naked DNA or complexed to lipid carriers, is described in U.S. Pat. No. 6,806,084.

The AONs for use in the methods or uses of the present invention is to be administered in an effective amount. The phrase ‘therapeutically effective amount’ or ‘effective amount’ generally refers to an amount of an AON, a pharmaceutically acceptable salt, polymorph or prodrug thereof as described herein that (i) treats the particular disease, condition, or disorder, (ii) attenuates, ameliorates, or eliminates one or more symptoms of the particular disease, condition, or disorder, or (iii) delays the onset of one or more symptoms of the particular disease, condition, or disorder described herein. Undesirable effects, e.g. side effects, are sometimes manifested along with the desired therapeutic effect, hence, a practitioner balances the potential benefits against the potential risks in determining what is an appropriate “effective amount”.

The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation. In one aspect, the dose administered to a subject is any dose that reduces viral load.

The phrase “pharmaceutically acceptable” refers to molecular entities and compositions that are physiologically tolerable and do not typically produce an allergic or similarly untoward reaction, such as gastric upset and the like, when administered to a patient. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or saline solutions and aqueous dextrose and glycerol solutions are preferably employed as carriers, particularly for injectable solutions. Suitable pharmaceutical carriers are described in Martin, Remington's Pharmaceutical Sciences, 18th Ed., Mack Publishing Co., Easton, PA, (1990).

Treatment may be monitored, e.g., by general indicators of disease known in the art. The efficacy of an in vivo administered AONs for use in the methods of the present invention may be determined from biological samples (tissue, blood, urine etc.) taken from a subject prior to, during and subsequent to administration of the AON. Assays of such samples include (1) monitoring the presence or absence of heteroduplex formation with target and non-target sequences, using procedures known to those skilled in the art, e.g., an electrophoretic gel mobility assay, (2) monitoring the amount of a mutant mRNA in relation to a reference normal mRNA or protein as determined by standard techniques such as RT-PCR, Northern blotting, ELISA or Western blotting.

The methods of the invention extends also to a combination of two or more AONs capable of binding to a selected target to induce exon exclusion in a RAGE gene transcript. The combination may be a cocktail of two or more AONs, a construct comprising two or more or two or more AONs joined together for use in an AON-based therapy. The combination of AONs is preferably a combination of SEQ ID NO: 11 and 10, or SEQ ID NO: 11 and 13.

Kits are also provided for use in the methods of the invention, in particular to treat, prevent or ameliorate a disease or condition associated with RAGE expression in a patient, which kit comprises at least an AON as described herein together with instructions for its use, wherein the instructions for use describe a method of the invention.

The contents of the kit can be lyophilized and the kit can additionally contain a suitable solvent for reconstitution of the lyophilized components. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale for human administration.

As used herein the term “derived” and “derived from” shall be taken to indicate that a specific integer may be obtained from a particular source albeit not necessarily directly from that source.

As used herein, the singular forms “a,” “an” and “the” include plural references unless the context clearly dictates otherwise.

Other than in the operating example, or where otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the specification and claims are approximations that may vary depending upon the desired properties sought to be obtained by the methods of the present invention. Hence “about 80%” means “about 80%” and also “80%”. At the very least, each numerical parameter should be construed in light of the number of significant digits and ordinary rounding approaches.

Notwithstanding that the numerical ranges and parameters setting forth the broad scope of the methods of the present invention are approximations, the numerical values set forth in the specific examples are reported as precisely as possible. Any numerical value, however, inherently contains certain errors necessarily resulting from the standard deviation found in their respective testing measurements

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

It will be understood that the invention disclosed and defined in this specification extends to all alternative combinations of two or more of the individual features mentioned or evident from the text or drawings. All of these different combinations constitute various alternative aspects of the invention.

### EXAMPLES

### Example 1—Materials and Methods

### Animals

Pathogen-free male C57BL/6 mice (8-10 weeks old) were used in the present studies. The animals were housed in sterile passive micro-isolators at a constant 20° C. temperature on a 12-h day/night cycle and fed irradiated Barastoc mouse feed with irradiated tap water allowed ad libitum. Mouse weights were monitored throughout the experiment. Throughout the study animals were given access to mouse chow and water ad libitum. All experiments were approved by local animal ethics committee and conducted in accordance with the Guide for the Care and Use of Laboratory Animals published by the US National Institutes of Health (NIH Publication No. 85-23, revised 1996).

### Bleomycin-Induced Pulmonary Fibrosis Mouse Model

The present studies aim to demonstrate changes in pathogenic markers or pathology in bleomycin-induced pulmonary fibrosis, an experimental model of pulmonary fibrosis. 72 female C57BL/6 mice (n=6/groups) were randomised to receive treatment with saline or ASO 6713 (3 or 10 mg/kg; SEQ ID NO: 33) via intralobular instillation 7 days prior intralobular instillation of bleomycin or vehicle control (day −7) and followed for either 7 days after bleomycin (Inflammatory phase) or 14 days (active profibrotic phase) after which they were humanely killed. ASO 6713 was used to test the therapeutic potential of an AON that is complementary to a pre-mRNA target region that has a 5′-most nucleotide at position 88 or 114 of exon 10 or that is between nucleotide positions 88 to 114 of exon 10.

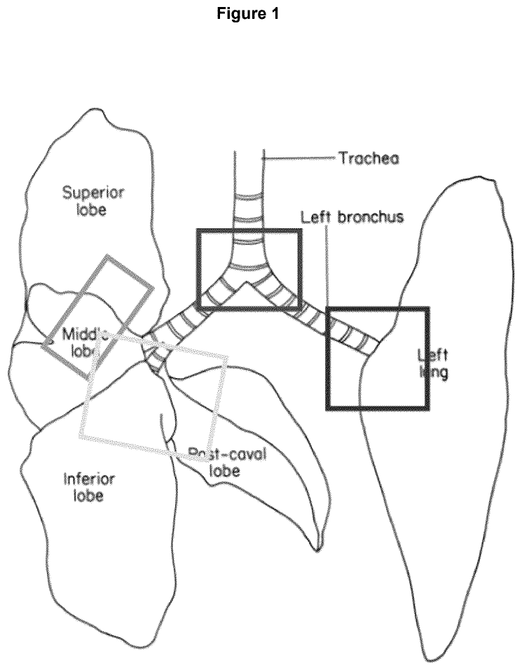

Intralobular instillation was performed under general anaesthesia and delivered specifically into the left main bronchus of the mice (FIG. 1A, red box). ASO 6713 and saline was provided by RAGE Biotech Pty Ltd and researchers were blinded to doses provided to investigators.

C57BL/6 mice (n=6/group) were randomised to the following 8 groups:

| Group | Treatment | Endpoint | Group Aim |
|---|---|---|---|
| A | Vehicle on day −7 and PBS on day 0 | Day 7 | Control-1 |
| B | Vehicle on day −7 and PBS on day 0 | Day 14 | Control-2 |
| C | Vehicle on day −7 and bleomycin on | Day 7 | Untreated-1 |
| | day 0 | | |
| D | Vehicle on day −7 and bleomycin on | Day 14 | Untreated-2 |
| | day 0 | | |
| E | ASO 6713 (3 mg/kg) on day −7 and | Day 7 | ASO 6713- |
| | bleomycin on day 0 | | low-1 |
| F | ASO 6713 (3 mg/kg) on day −7 and | Day 14 | ASO 6713- |
| | bleomycin on day 0 | | low-2 |
| G | ASO 6713 (10 mg/kg) on day −7 and | Day 7 | ASO 6713- |
| | bleomycin on day 0 | | high-1 |
| H | ASO 6713 (10 mg/kg) on day −7 and | Day 14 | ASO 6713- |
| | bleomycin on day 0 | | high-2 |

Mice weight did not significantly vary between groups from day 1 to day 10 following treating with bleomycin or vehicle (data not shown).

### Study Endpoints

Flow cytometry: Phenotyping and characterization of various cell types in BALF were performed by flow cytometry. Lung mouse tissue was digested using collagenase IV (BioConcept, Worthington, Lakewood, NY) in Dulbecco's Modified Eagle Media (DMEM) for 45 min at 37° C. Samples were then filtered through a 70-μm cell strainer and washed in DMEM 10% FCS. Cells were stained with antibodies to CD45 FITC (Biolegend, clone 30-F11), CD45 BUV395 (BD Biosciences, clone 30-F11), CD3 BUV737 (Biolegend, clone 145-2C11), CD19 BV650 (BD Biosciences, clone 1D3), CD8 BV786 (BD Biosciences, clone H35-17.2), CD4 BV711 (BD Biosciences, clone GK1.5), CD11c APCCy7 (Biolegend, N418), Siglec-F PE-CF594 (BD Biosciences, clone E50-2440), CD11b PB (Biolegend, clone M1/70), Gr-1 PE (Biolegend, clone RB6-8C5), Ly-6C FITC (BD Biosciences, clone AL-21), Ly-6G AF647 (Biolegend, clone 1A8). All cells were acquired on a Fortessa (BD Biosciences) and analysis was performed using FlowJo software (Tree Star, Ashland, OR).

Quantification of the BALF cytokines IL-6 and CCL2 was performed using the LEGENDplex™ Mouse Inflammation Panel (Biolegend, #740446) followed by flow cytometry (Fortessa BD Biosciences).

### Example 2—Segmental Broncho-Alveolar Lavage Fluid (BALF) Analysis During Inflammatory Phase and Profibrotic Phase of the Bleomycin-Induced Pulmonary Fibrosis Mouse Model

In this experiment, mice (6 per group) were treated with saline or ASO 6713 (3 or 10 mg/kg) via intralobular instillation 7 days prior to intralobular instillation of bleomycin or vehicle (control). Segmental broncho-alveolar lavage fluid (BALF) was analysed in the mice 7 days or 14 days following bleomycin or vehicle control instillation.

At day 7 following bleomycin treatment without ASO 6713 intervention, an inflammatory response can be visibly seen on the left lung of the mice. At day 14, the lungs show signs of profibrotic scarring indicative of pulmonary fibrosis (data not shown).

### Example 3—Analysis of Nucleated Hematopoietic Cells in a Bleomycin-Induced Pulmonary Fibrosis Mouse Model with or without ASO 6713 Treatment

Mice were treated according to the same method as described in Example 2 above. BALF was analysed for CD45 expression using flow cytometry to determine the number of CD45+ within each treatment group.

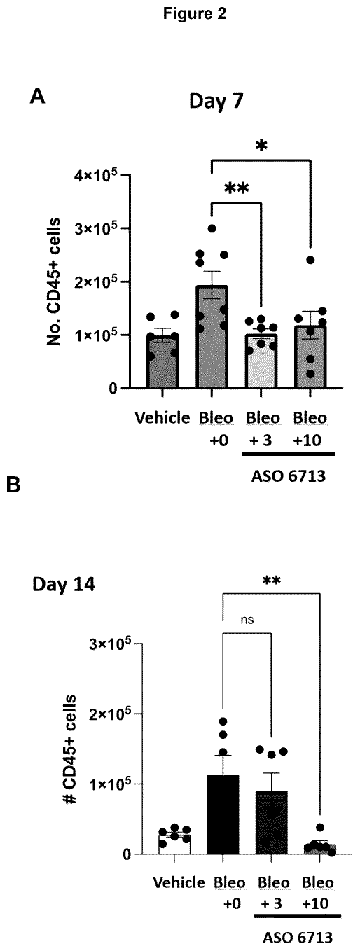

Both low dose (3 mg/kg) and high dose (10 mg/kg) ASO 6713 significantly reduced CD45+ cells 7 days following treatment with bleomycin compared to the ASO 6713 untreated bleomycin group (FIG. 2A). This effect was more pronounced at day 14 for the high dose ASO 6713 treatment group (FIG. 2B). The low dose, however, was not statistically different to the ASO 6713 untreated group at day 14.

These results suggest that ASO 6713 treatment is capable of preventing an increase in CD45+ leukocytes which is typically associated with inflammatory responses.

### Example 4—Analysis of Interstitial Macrophages and T Cells in a Bleomycin-Induced Pulmonary Fibrosis Mouse Model with or without ASO 6713 Treatment

In this experiment, mice (6 per group) were treated with saline or ASO 6713 (3 or 10 mg/kg) via intralobular instillation 7 days prior to intralobular instillation of bleomycin or vehicle (control). Segmental broncho-alveolar lavage fluid (BALF) was analysed in the mice 7 days following bleomycin or vehicle control instillation. BALF was analysed for interstitial (non-alveolar) macrophages (Ly6C+ monocytes), CD4+ T cells and CD8+ T cells using flow cytometry.

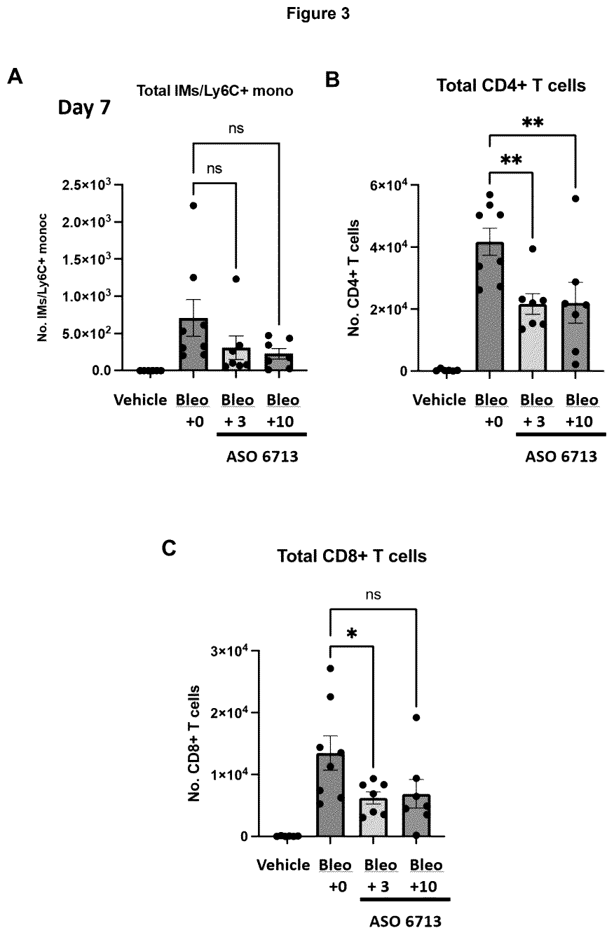

Both low dose (3 mg/kg) and high dose (10 mg/kg) ASO 6713 treatment significantly reduced CD4+ T cells numbers 7 days following treatment with bleomycin compared to the ASO 6713 untreated bleomycin group (FIG. 3B). Similarly, low dose ASO 6713 treatment significantly reduced CD8+ T cells numbers compared to the untreated group, however, the high dose ASO 6713 treatment was not significantly different though shared a decreased trend with the low dose treatment (FIG. 3C).

Although neither the high or low dose ASO 6713 significantly reduced the number of interstitial macrophages in the BALF, there was a consistent trend towards reduced cell numbers compared to the ASO 6713 untreated group (FIG. 3A).

Overall, these results suggest that ASO 6713 treatment may reduce activation of an immune response following bleomycin treatment in the lungs of mice.

### Example 5—Analysis of B Cells, Eosinophils and Neutrophils in a Bleomycin-Induced Pulmonary Fibrosis Mouse Model with or without ASO 6713 Treatment

Mice were treated according to the same method as described in Example 4 above. BALF was analysed for B cells (CD19+), eosinophils and neutrophils using flow cytometry.

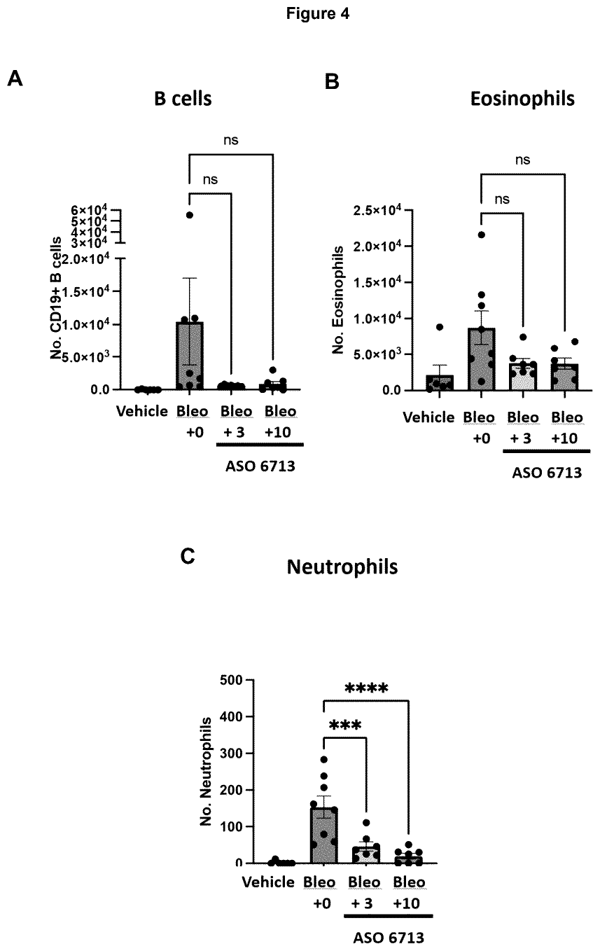

Both low dose (3 mg/kg) and high dose (10 mg/kg) ASO 6713 treatment significantly reduced neutrophil cell numbers 7 days following treatment with bleomycin compared to the ASO 6713 untreated bleomycin group (FIG. 4C).

Neither the high or low dose ASO 6713 significantly reduced the number of B cells or eosinophils in the BALF, however, there was a consistent trend towards reduced cell numbers compared to the ASO 6713 untreated group (FIG. 4A&B).

Consistent with the results from the previous Example, these results suggest that ASO 6713 treatment may reduce activation of an immune response following bleomycin treatment in the lungs of mice.

### Example 6—Analysis of Activated Macrophages, Interstitial Macrophages and Dendritic Cells in a Bleomycin-Induced Pulmonary Fibrosis Mouse Model with or without ASO 6713 Treatment

In this experiment, mice (6 per group) were treated with saline or ASO 6713 (3 or 10 mg/kg) via intralobular instillation 7 days prior to intralobular instillation of bleomycin or vehicle (control). Segmental broncho-alveolar lavage fluid (BALF) was analysed in the mice 14 days following bleomycin or vehicle control instillation. BALF was analysed for activated macrophages (CD11b+), interstitial (non-alveolar) macrophages (Ly6C+) and dendritic cells (CD11c+) using flow cytometry.

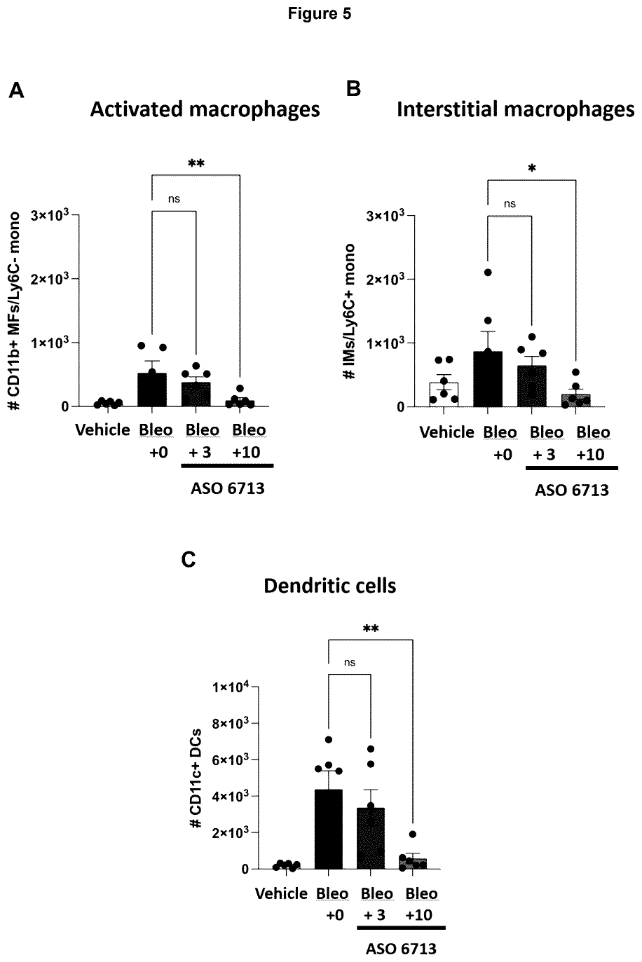

High dose (10 mg/kg) ASO 6713 treatment significantly reduced activated macrophages, interstitial macrophages and dendritic cell numbers 14 days following treatment with bleomycin compared to the ASO 6713 untreated bleomycin group (FIG. 5). However, the low dose (3 mg/kg) ASO 6713 treatment was not significantly different though shared a decreased trend with the high dose treatment.

Overall, these results suggest that ASO 6713 treatment continues to protect mice against an inflammatory response up into the profibrotic phase.

### Example 7—Analysis of CD4+ T Cells, CD8+ T Cells and B Cells in a Bleomycin-Induced Pulmonary Fibrosis Mouse Model with or without ASO 6713 Treatment

Mice were treated according to the same method as described in Example 6 above. BALF was analysed for CD4+ T cells, CD8+ T cells and B cells (CD19) using flow cytometry.

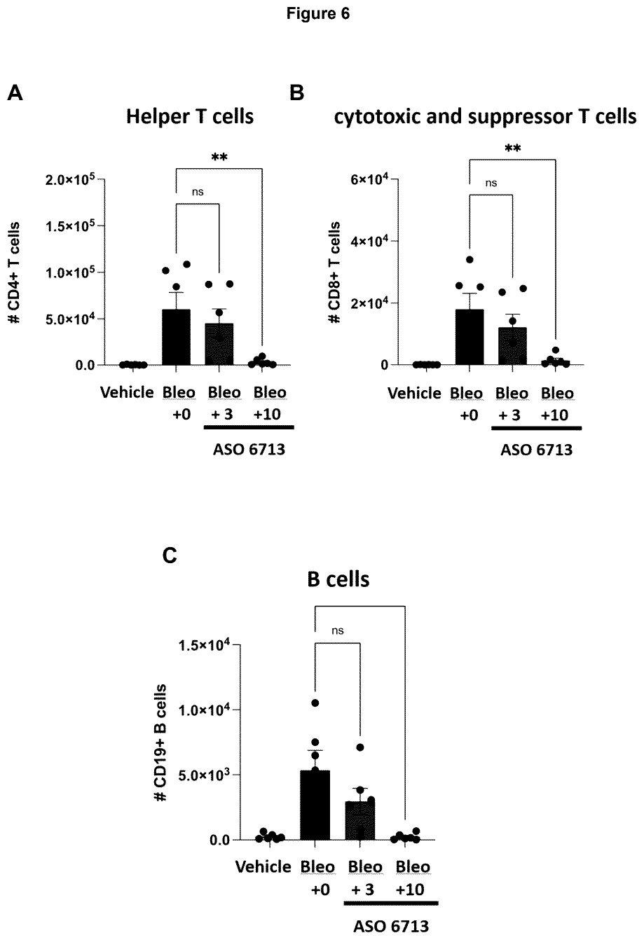

High dose (10 mg/kg) ASO 6713 treatment significantly reduced CD4+ T cells, CD8+ T cells and B cells numbers 14 days following treatment with bleomycin compared to the ASO 6713 untreated bleomycin group (FIG. 6). However, the low dose (3 mg/kg) ASO 6713 treatment was not significantly different though shared a decreased trend with the high dose treatment.

Consistent with the results from the previous Example, these results suggest that the ASO 6713 treatment continues to protect mice against an inflammatory response up into the profibrotic phase.

### Example 8—Cytokine Levels in Segmental Bronchoalveolar Fluid from the Left Lobe of a Bleomycin-Induced Pulmonary Fibrosis Mouse Model with or without ASO 6713 Treatment

In this experiment, mice (6 per group) were treated with saline or ASO 6713 (3 or 10 mg/kg) via intralobular instillation 7 days prior to intralobular instillation of bleomycin or vehicle (control). Segmental broncho-alveolar lavage fluid (BALF) was analysed in the mice 14 days following bleomycin or vehicle control instillation. BALF was analysed for IL6 and CCL2 using an LEGENDplex™ Mouse Inflammation Panel (Biolegend, #740446) followed by flow cytometry.

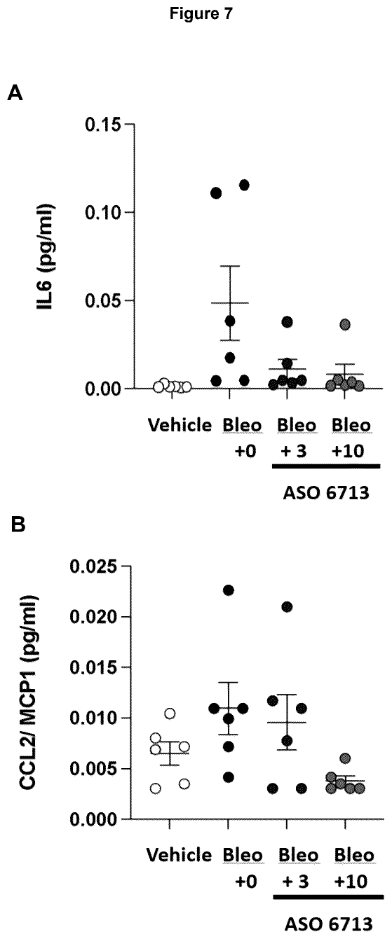

Although not statistically significant in the present, ASO 6713 treatment appeared to reduce both IL6 and CCL2 expression compared to ASO 6713 untreated mice in cells obtained from BALF samples (FIG. 7).

### Example 9—Lymphocytic Nodules in a Bleomycin-Induced Pulmonary Fibrosis Mouse Model Treated with or without ASO 6713

In this experiment, mice (5-7 per group) were treated with saline or ASO 6713 (3 or 10 mg/kg) via intralobular instillation 7 days prior to intralobular instillation of bleomycin or vehicle (control). Lymphocytic nodules were counted (n/field) in the left lung of mice 7 days or 14 days following bleomycin or vehicle control instillation.

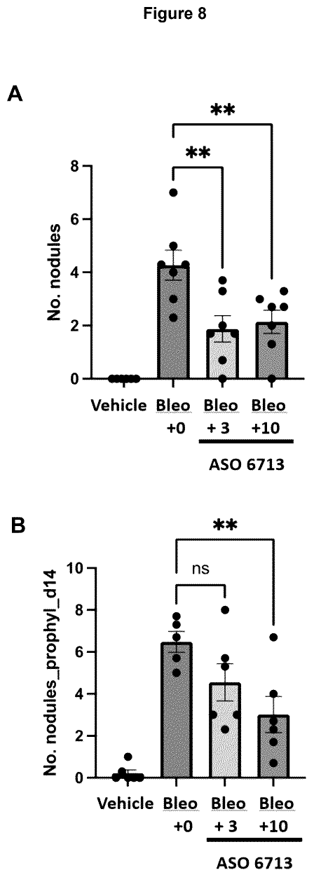

Low dose (3 mg/kg) and high dose (10 mg/kg) ASO 6713 treatment decreased the number lymphocytic nodules in mice lungs at 7 days following bleomycin treatment compared to the ASO 6713 untreated group (FIG. 8A). However, at day 14 only the high dose ASO 6713 still presented with a statistically significant reduction of lymphocytic nodules (FIG. 8B).

### Example 10—Analysis of the Severity of Mouse Lung Injury 14 Days Following Treatment with Bleomycin or Vehicle, with or without ASO 6713 Treatment

In this experiment, mice (5-6 per group) were treated with saline or ASO 6713 (3 or 10 mg/kg) via intralobular instillation 7 days prior to intralobular instillation of bleomycin or vehicle (control). Mice lungs were scored according to a mouse-adapted Ashcroft score 14 days following bleomycin or vehicle control instillation.

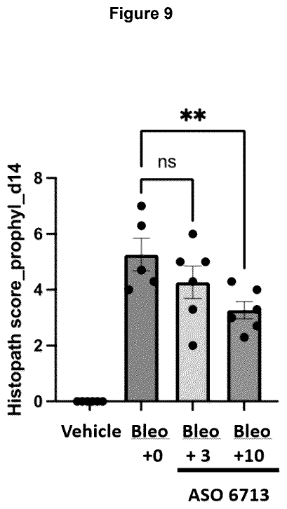

High dose (10 mg/kg) ASO 6713 treatment showed a significant reduction in histopathology scoring compared to the ASO 6713 untreated group (FIG. 9).

These results suggests that ASO 6713 treatment can protect against pulmonary fibrosis injury in mice.

1. A method of treating or preventing a condition of the airway or lung involving fibrosis in a subject in need thereof, the method comprising administering to the subject an antisense oligonucleotide (AON) that promotes the production of endogenous soluble RAGE and/or reduces the production of membrane bound RAGE (mRAGE), thereby treating or preventing a condition of the airway or lung involving fibrosis in a subject.

2. A method of alleviating or ameliorating a symptom of a condition of the airway or lung involving fibrosis in a subject in need thereof, the method comprising administering to the subject in need thereof an antisense oligonucleotide (AON) that promotes the production of endogenous soluble RAGE and/or reduces the production of membrane bound RAGE (mRAGE), thereby alleviating or ameliorating a symptom of a condition of the airway or lung involving fibrosis in the subject.

3. Use of an antisense oligonucleotide (AON) that promotes the production of endogenous soluble RAGE and/or reduces the production of membrane bound RAGE (mRAGE) in the manufacture of a medicament for the treatment or prevention of a condition of the airway or lung involving fibrosis in a subject in need thereof.

4. A method or use according to any one of the preceding claims, wherein the AON that promotes the production of endogenous soluble RAGE and/or reduces the production of membrane bound RAGE (mRAGE) is administered directly to the airway and/or lungs.

5. A method or use according to any one of the preceding claims, wherein the administration of the AON promotes the production of endogenous soluble RAGE and/or reduces the production of membrane bound RAGE (mRAGE) in the total respiratory tract, lower respiratory tract or upper respiratory tract.

6. A method or use according to any one of the preceding claims, wherein the method promotes the production of endogenous soluble RAGE and/or reduces the production of membrane bound RAGE (mRAGE) in the lower respiratory tract, preferably the lung.

7. A method or use according to any one of the preceding claims, wherein the antisense oligonucleotide (AON) that promotes the production of endogenous soluble RAGE and/or reduces the production of membrane bound RAGE (mRAGE) is administered via inhalation.

8. A method or use according to any one of the preceding claims, wherein the antisense oligonucleotide (AON) that promotes the production of endogenous soluble RAGE and/or reduces the production of membrane bound RAGE (mRAGE) is administered via intranasal administration.

9. A method or use according to any one of the preceding claims, wherein the condition of the airway or lung involving fibrosis is pulmonary fibrosis.

10. A method or use according to any one of the preceding claims, wherein the pulmonary fibrosis is idiopathic pulmonary fibrosis, familial pulmonary fibrosis, pulmonary fibrosis caused by sarcoidosis, pulmonary fibrosis caused by silicosis, pulmonary fibrosis caused by asbestosis, pulmonary fibrosis caused by coal worker's pneumoconiosis, pulmonary fibrosis caused by carbon pneumoconiosis, pulmonary fibrosis caused by hypersensitivity pneumonitides, pulmonary fibrosis caused by inhalation of inorganic dust, pulmonary fibrosis caused by an infectious agent, pulmonary fibrosis caused by inhalation of noxious gases, aerosols, chemical dusts, fumes or vapors, drug-induced interstitial lung disease.

11. A method or use according to any one of the preceding claims, wherein the pulmonary fibrosis is idiopathic pulmonary fibrosis.

12. The method of any one of claims 1 to 11, wherein the AON promotes the production of endogenous soluble RAGE by promoting the inclusion of exon 9b and/or the exclusion (e.g. skipping) of exon 10.

13. The method of any one of claims 1 to 11, wherein AON promotes splicing in the RAGE pre-mRNA resulting in the inclusion of exon 9b and/or skipping of exon 10.

14. The method of claim 12 or 13, wherein administration of the AON results in an increase of the level of RAGE_v1, preferably an increase in the level of RAGE_v1 mRNA in one or more tissues of the respiratory tract.

15. The method of any one of claims 1 to 14, wherein the AON is administered to the total respiratory tract, the upper respiratory tract or the lower respiratory tract.

16. The method of any one of claims 1 to 15, wherein the AON is administered as an aerosol.

17. The method of any one of claims 1 to 16, wherein the AON is administered in a single dose.

18. The method of any one of claims 1 to 17, wherein the AON is administered via the same route of administration in the first, second or further administrations.

19. The method of any one of claims 1 to 18, wherein the same AON is administered in each of the first, second or further administrations.

20. The method of any one of claims 1 to 19, wherein the AON is an AON of 10 to 50 nucleotides comprising a targeting sequence complementary to a region near or within an intron of the RAGE pre-mRNA, optionally, the AON is 10 to 50 nucleotides comprising a targeting sequence complementary or adjacent to a splice site of the RAGE pre-mRNA.

21. The method of any one of claims 1 to 20, wherein, the AON is 10 to 50 nucleotides comprising a targeting sequence complementary to RAGE pre-mRNA which modulates secondary structure of said mRNA to influence splice site selection.

22. The method of any one of claims 1 to 21, wherein the AON is an isolated or purified AON for inducing exclusion (also known as skipping) of one or more exonic sequences in the RAGE gene transcript or part thereof.

23. The method of any one of claims 1 to 22, wherein the AON is an isolated or purified AON for inducing retention of intronic sequences in the RAGE gene transcript or part thereof.

24. The method of any one of claims 1 to 23, wherein the AON comprises at least one modified nucleotide.

25. The method of any one of claims 1 to 24, wherein the AON is chemically-modified to prevent degradation of the pre-mRNA-AON complex, preferably wherein the chemical modification is selected from the group consisting of: phosphorodiamidate morpholino oligomers (PMO), 2′ O-methyl phosphorothioate oligonucleotides (2OMe), and 2′-O-methoxyethyl phosphorothioate oligonucleotides (2′-MOE), locked nucleic acid (LNA) modified AONs, thermostable twisted intercalating nucleic acid (TINA) and peptide nucleic acids (PNAs).

26. The method of any one of claims 1 to 25, wherein the AON is conjugated to moieties to increase its delivery, preferably cell-penetrating peptides (CPPs), vivo-morpholinos (VMO) or peptide phosphorodiamidate morpholino oligomers (PPMO).

27. The method of any one of claims 1 to 26, wherein the AON comprises, consists essentially of or consists of a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or 100% complementary to a target region of exon 10 of RAGE pre-mRNA over the entirety of the antisense oligonucleotide.

28. The method of claim 27, wherein the 5′-most nucleotide of the target region is nucleotide position 88 or 114 of exon 10 or is between nucleotide positions 88 to 114 of exon 10.

29. The method of any one of claims 1 to 28, wherein the AON is 8 to 40 nucleotides in length, 15 to 25 nucleotides in length or 18 nucleotides in length.

30. The method of any one of claims 1 to 29, wherein the AON is selected from the group comprising the sequences set forth in any of Tables 1a-1d.

31. The method of any one of claims 1 to 30, wherein the AON comprises the nucleotide sequence as set forth in any one of SEQ ID NO: 1-31 and 33 or a nucleotide sequence at least 85%, 90% or 95% identical thereto.

32. The method of claim 31, wherein the AON comprises the nucleotide sequence as set forth in SEQ ID NO: 11, 18, 19, or 20, or a nucleotide sequence at least 85%, 90% or 95% identical thereto.


SEQUENCE LISTING (33 sequences)

SEQ ID NO: 1 - RNA, 25 nt, synthetic construct
  source 1..25: mol_type = other RNA
cctcctcgcctggttctggaagaca

SEQ ID NO: 2 - RNA, 20 nt, synthetic construct
  source 1..20: mol_type = other RNA
ttggcccctcctcgcctggt

SEQ ID NO: 3 - RNA, 20 nt, synthetic construct
  source 1..20: mol_type = other RNA
ctgcagttggcccctcctcg

SEQ ID NO: 4 - RNA, 20 nt, synthetic construct
  source 1..20: mol_type = other RNA
tcaaacccctcacctgcagt

SEQ ID NO: 5 - RNA, 25 nt, synthetic construct
  source 1..25: mol_type = other RNA
cctgcagttggcccctcctcgcctg

SEQ ID NO: 6 - RNA, 25 nt, synthetic construct
  source 1..25: mol_type = other RNA
cccctcacctgcagttggcccctcc

SEQ ID NO: 7 - RNA, 17 nt, synthetic construct
  source 1..17: mol_type = other RNA
gcagttggcccctcctc

SEQ ID NO: 8 - RNA, 25 nt, synthetic construct
  source 1..25: mol_type = other RNA
cacctgcagttggcccctcctcgcc

SEQ ID NO: 9 - RNA, 25 nt, synthetic construct
  source 1..25: mol_type = other RNA
atcctcccacagagcctgtacggag

SEQ ID NO: 10 - RNA, 25 nt, synthetic construct
  source 1..25: mol_type = other RNA
ccctgatcctcccacagagcctgta

SEQ ID NO: 11 - RNA, 25 nt, synthetic construct
  source 1..25: mol_type = other RNA
ggcgttgccgcctttgccacaagat

SEQ ID NO: 12 - RNA, 25 nt, synthetic construct
  source 1..25: mol_type = other RNA
ctcacctctcctctcctcggcgttg

SEQ ID NO: 13 - RNA, 25 nt, synthetic construct
  source 1..25: mol_type = other RNA
ctccactcacctctcctctcctcgg

SEQ ID NO: 14 - RNA, 20 nt, synthetic construct
  source 1..20: mol_type = other RNA
agcagggcggctgtccccag

SEQ ID NO: 15 - RNA, 25 nt, synthetic construct
  source 1..25: mol_type = other RNA
gccacaagatgaccccaatgagcag

SEQ ID NO: 16 - RNA, 25 nt, synthetic construct
  source 1..25: mol_type = other RNA
cctttgccacaagatgaccccaatg

SEQ ID NO: 17 - RNA, 25 nt, synthetic construct
  source 1..25: mol_type = other RNA
tgccgcctttgccacaagatgaccc

SEQ ID NO: 18 - RNA, 25 nt, synthetic construct
  source 1..25: mol_type = other RNA
tcctcggcgttgccgcctttgccac

SEQ ID NO: 19 - RNA, 13 nt, synthetic construct
  source 1..13: mol_type = other RNA
tttgccacaagat

SEQ ID NO: 20 - RNA, 20 nt, synthetic construct
  source 1..20: mol_type = other RNA
ccgcctttgccacaagatga

SEQ ID NO: 21 - RNA, 25 nt, synthetic construct
  source 1..25: mol_type = other RNA
tttcttgttgaccatccccccagtc

SEQ ID NO: 22 - RNA, 25 nt, synthetic construct
  source 1..25: mol_type = other RNA
ccatccccccagtcacatgtgttgg

SEQ ID NO: 23 - RNA, 25 nt, synthetic construct
  source 1..25: mol_type = other RNA
cctcctcgcctggttctggaagaca

SEQ ID NO: 24 - RNA, 20 nt, synthetic construct
  source 1..20: mol_type = other RNA
ctggcccctcatcgccggtt

SEQ ID NO: 25 - RNA, 20 nt, synthetic construct
  source 1..20: mol_type = other RNA
cttcagctggcccctcatcg

SEQ ID NO: 26 - RNA, 20 nt, synthetic construct
  source 1..20: mol_type = other RNA
tccagtccctcaccttcagc

SEQ ID NO: 27 - RNA, 25 nt, synthetic construct
  source 1..25: mol_type = other RNA
tgggttgtcgttttcgccacaggat

SEQ ID NO: 28 - RNA, 23 nt, synthetic construct
  source 1..23: mol_type = other RNA
actactcccaggcctcccaggat

SEQ ID NO: 29 - RNA, 22 nt, synthetic construct
  source 1..22: mol_type = other RNA
gcagggctactactcccaggca

SEQ ID NO: 30 - RNA, 25 nt, synthetic construct
  source 1..25: mol_type = other RNA
tctcactcacctctcctcacgcctg

SEQ ID NO: 31 - RNA, 25 nt, synthetic construct
  source 1..25: mol_type = other RNA
cccagactcacccacagagcctgta

SEQ ID NO: 32 - RNA, 127 nt, Homo sapiens
  source 1..127: mol_type = mRNA
gctctgtgggaggatcagggctgggaactctagccctggccctggggatcctgggaggcctggggacagccgccctgctcattggggtcatcttgtggcaaaggcggcaacgccgaggagaggagag

SEQ ID NO: 33 - RNA, 18 nt, synthetic construct
  source 1..18: mol_type = other RNA
tcgttttcgccacaggat